SEQUENCE LISTING (11 sequences)

SEQ ID NO: 1 - DNA, 2601 bp, Homo sapiens
  CDS (1147)...(1407)
cgccactgtgttggaattcggcacgagggggatctggaagggccaatagaagatactcagcactaagagacctttggactcagaccagaacttacaccattcattcattcagtcggtgaaaatgtattgaccactgtatcaaccaggattgtgacacaaaaacagatggcacactcaaaagaggataattcaagaagggcttctttaagggactatttcccaagatgggaatggaggggaacctgcagggctagtgtcctaccctccagcaggcagcagctaattcctgaggggataaggacgtggttgcgaggacatggagggaaagttctacagaggaggcacagtgggcttcaggaacaccctgcttgagaggcctgtgagaggtggggaatcaatacctgacctcgctctccttccatctctccccaacccacaggggttggtgtgggccccacaggcgagcctcccggggagagaagtggagagaggacctggagggccagtagaaggtatgcacacaagtatctacaaggcaccaggcattttttgagcatttgggatttgtcagcaaacaagtcagacaaaaaaccttgctctggtggagggaacattctagcaaaggaaggcaaatgacaagcagtaagtacaataatcaagtaaaatagataccaggttagagagtgataaatgcgatgggaaaaaatacagcaggtgaaggaggttggagagtagggggtggagggcccacgcagcacttgtccttcaccctggaggggatctgttacatgccccagattgctggtcccctagaaatgttactgaggcagcctctgcatttttgcagggattgttttctactgtttgacattcacgtaacctcctaacgctgtctggggaagatgctaccccctgctctccccgtctttcctgcactctcagcaatgggatgggctgactgatgccctgtgggctggaaagctgaccacagttgctgcagaccagaccccctcacatagtgagtgctgggctgaggaatccaggagagcccgaggggggacactgaaggtgtatcgttggccctgccagctgcaagtgaactgcttctgatgaattttaatagggagaaagaagtatttgctaagaatggcaatcctgatgctcagccttcaactcatcttgttattaataccatcaatatcccatgaggctcataaaacgagtctttcttcttggaaacatgaccaagattgggcaaacgtctccaacatgactttcagcaacggaaaactaagagtcaaaggcatttattaccggaatgccgacatttgctctcgacatcgcgtaacctcagcaggcctaactctgcaggaccttcagctatggtgtaatttgagaatcattcactgagcatcaactatgtaaccagcattgggttgggtgccagagatccaaagctaagacaccaaaacctgctctccaggaaacgagaggctgagaagagggccagcaggtgtctgtcagtacttggagccgtgagagcagggagtgggtgctgggctgaggaaccagaggtaatggccctggggacgcccgggaagagatgagttttgaggcaaagaattgttaacatacaagataatcaaagcacgaaggctctgatgcgtgataaaataatcatttctcaaaacaggaagatgagaactgcatttcgagttgtatcacttggcacacaatacttgcaatctgtgtgctgtaattacagtgtttcttcactctaagtgcatctgactgatactagcataacaaaagacgtgattgcagtagtgtttttcttttacttcatttgttaaacagtgcagaaatccaaataacaacattctcaacagcaaacagaatctctgtcatttgagaaggttttgctacgctacagaatgcctgtgtttggaaaaacagagaaaaaggtttttagcgggttccactaagcacagtattctatctgcttggtatacacgatcaaaaaataacttaacctttgtctagggaaagtctttaaggtagctcttactgcatatcttcactatatgtacacagacaccatatttatatattatatatttatataagacatgtatgtacacatttacagaccttcaaaaatatattgcacttatatacaatgcagctttatcttaactgatttcatactgtaacccattaaaattcttcatgagaaaggcagttgatatgtccgagaaagtcgcaaaggaagatttcagtaacatgccctgtttagtaaacatctggtggagagtgagggggtaagggagagagggctccccccgtactcttcaggtgcgctcccgctaacgtgaggcagtggagttgtactgaattcaggcaagggcacgaaatccttaaagccaagcctatcgccttttctgacttctgctagcgagcaggcccacgacactgtaggcacaaggcagagatcccaatattttgataaataacgtagcagatgtcctaaagcttcccagagactctgttaactcttggaataaagttttcactttaaatcctgtatatatcaggaaattc

SEQ ID NO: 2 - protein, 87 aa, Homo sapiens
MAILMLSLQLILLLIPSISHEAHKTSLSSWKHDQDWANVSNMTFSNGKLRVKGIYYRNADICSRHRVTSAGLTLQDLQLWCNLRIIH

SEQ ID NO: 3 - DNA, 261 bp, Artificial Sequence
  Degenerate nucleotide
atggcnathytnatgytnwsnytncarytnathytnytnytnathccnwsnathwsncaygargcncayaaracnwsnytnwsnwsntggaarcaygaycargaytgggcnaaygtnwsnaayatgacnttywsnaayggnaarytnmgngtnaarggnathtaytaymgnaaygcngayathtgywsnmgncaymgngtnacnwsngcnggnytnacnytncargayytncarytntggtgyaayytnmgnathathcay

SEQ ID NO: 4 - protein, 20 aa, Homo sapiens
CSRHRVTSAGLTLQDLQLWC

SEQ ID NO: 5 - protein, 11 aa, Artificial Sequence
  peptide motif
XXXXXXGXXXX

SEQ ID NO: 6 - protein, 11 aa, Homo sapiens
GKLRVKGIYYR

SEQ ID NO: 7 - protein, 9 aa, Homo sapiens
AGLTLQDLQ

SEQ ID NO: 8 - DNA, 22 bp, Artificial Sequence
  oligonucleotide ZC40264
ctaagaatggcaatcctgatgc

SEQ ID NO: 9 - DNA, 22 bp, Artificial Sequence
  oligonucleotide ZC40267
cccaatgctggttacatagttg

SEQ ID NO: 10 - DNA, 24 bp, Artificial Sequence
  oligonucleotide ZC23674
cgaaacacaaagttctatggtctc

SEQ ID NO: 11 - DNA, 22 bp, Artificial Sequence
  oligonucleotide ZC24776
gcaccacgatgaacacgaccaa

(19) United States
(12) Patent Application Publication
(10) Pub. No.: US 2003/0148466 A1
(43) Pub. Date: Aug. 7, 2003

(54) Secreted protein, ZTNF9

(76) Inventors: Brian A. Fox, Seattle, WA (US); Jane A. Gross, Seattle, WA (US)

Correspondence Address: Robyn Adams, ZymoGenetics, Inc, 1201 Eastlake Avenue East, Seattle, WA, 98102, US

(21) Appl. No.: 10/273,231

(22) Filed: Oct. 16, 2002

Related U.S. Application Data
(60) Provisional application No. 60/329,931, filed on Oct. 17, 2001.

(51) Int. Cl. C12P 21/02; C07H 21/04; C07K 14/525

(52) U.S. Cl. 435/69.5; 435/320.1; 435/325; 530/351; 536/23.5

(57) ABSTRACT

Novel tumor necrosis factor ligand polypeptides, polynucleotides encoding the polypeptides, and related compositions and methods are disclosed. The polypeptides may be used within methods relating to immune response, and may also be used in the development of immuno-regulatory therapeutics. Also provided are antibodies, binding proteins, agonists and antagonists of the ligand polypeptides.

### REFERENCE TO RELATED APPLICATIONS

This application is related to Provisional Applications 60/329,931 filed on Oct. 17, 2001. Under 35 U.S.C. § 119(e)(1), this application claims benefit of said Provisional Application.

### BACKGROUND OF THE INVENTION

Cellular interactions which occur during an immune response are regulated by members of several families of cell surface receptors and their respective ligands, including the tumor necrosis factor (TNF) family. Several members of this family regulate interactions between different hematopoietic cell lineages (Smith et al., The TNF Receptor Superfamily of Cellular and Viral Proteins: Activation, Costimulation and Death, 76:959-62, 1994; Cosman, Stem Cells 12:440-55, 1994). In general, the members of the TNF family mediate interactions between different hematopoietic cells, such as T cell/B cell, T cell/monocyte and T cell/T cell interactions. The result of this two-way communication can be stimulatory or inhibitory, depending on the target cell or the activation state. TNF ligands are involved in regulation of cell proliferation, activation and differentiation, including control of cell survival or death by apoptosis or cytotoxicity. Differences in TNF receptor (TNFR) distribution, kinetics of induction and requirements for induction, support the concept of a defined role for each of the TNF ligands in T cell-mediated immune responses.

The TNF ligand family is composed of a number of type II integral membrane glycoproteins. Members of this family, with the exception of nerve growth factor (NGF) and LT-α, contain an N-terminal cytoplasmic region, a single transmembrane region, a linker region and a 150 to 170 amino acid residue C-terminal receptor-binding domain. The tertiary structure of the C-terminal receptor-binding domain has been determined to be a β-sandwich. Members of this family, with the exception of NGF, share approximately 20% sequence homology within this extracellular receptor-binding domain, and little to no homology within the linker, transmembrane and cytoplasmic regions. The ligands within this family are biologically active as trimeric or multimeric complexes. This group includes TNF, LT-α, LT-β, CD27L, CD30L, CD40L, 4-IBBL, OX40L, FasL (Cosman, ibid.; Lotz et al., J. Leukoc. Biol. 60:1-7, 1996), TRAIL or apo-2 ligand (Wiley et al., Immunity 3:673-82, 1995), and TNF γ(WO 96/14328). The presence of a transmembrane region indicates that the ligands are membrane-associated. Soluble ligand forms have been identified for TNF, LT-α and FasL. It is not known whether a specific protease cleaves each ligand, releasing it from the membrane, or whether one protease serves the same function for all TNF ligand family members. TACE (TNF-alpha converting enzyme) has been shown to cleave TNF (Moss et al., Nature 385:733-36, 1997; Black et al., Nature 385:729-33, 1997). No other such enzyme is known.

The TNFR family is made up of type I integral membrane glycoproteins, including p75 NGFR, p55 TNFR-I, p75 TNFR-II, TNFR-RP/TNFR-III, CD27, CD30, CD40, 4-1BB, OX40, FAS/APO-1 (Cosman, ibid.; Lotz et al., ibid.), HVEM (Montgomery et al., Cell 87:427-36, 1996), WSL-1 (Kitson et al., Nature 384:372-75, 1996) also known as DR3 (Chinnaiyan et al., Science 274:990-92, 1996), DR4 (Pan et al., Science 276:111-13, 1997), a TNF receptor protein described in WO 96/28546 now known as osteoprotegerin (OPG, Simonet et al., Cell 89:309-19, 1997), CAR1, found in chicken (Brojatsch et al., Cell 87:845-55, 1996) plus several viral open reading frames encoding TNFR-related molecules. NGFR, TNFR-I, CD30, CD40, 4-1BB, DR3, DR4 and OX40 are mainly restricted to cells of the lymphoid/hematopoietic system.

The interaction of one member of the TNF ligand family, TNF, and its receptor, has been shown to be essential to a broad spectrum of biological processes and pathologies. In particular, the receptor-ligand pair has a variety of immunomodulatory properties, including mediating immune regulation, immunostimulation and moderating graft rejection. An involvement has also been demonstrated in inflammation, necrosis of tumors (Gray et al., Nature 312:721-24, 1984), septic shock (Tracy et al., Science 234:470-74, 1986) and cytotoxicity. TNF promotes and regulates cellular proliferation and differentiation (Tartalgia et al., J. Immunol. 151:4637-41, 1993. In addition, TNF and its receptor are also involved in apoptosis.

The X-ray crystallographic structures have been resolved for human TNF (Jones et al., Nature 388:225-28, 1989), LT-β (Eck et al., J. Biol. Chem. 267:2119-22, 1992), and the LT-β/TNFR complex (Banner et al., Cell 73:431-35, 1993). This complex features three receptor molecules bound symmetrically to one LT-β trimer. A model of trimeric ligand binding through receptor oligomerization has been proposed to initiate signal transduction pathways. The identification of biological activity of several TNF members has been facilitated through use of monoclonal antibodies specific for the corresponding receptor. These monoclonal antibodies tend to be stimulatory when immobilized and antagonistic in soluble form. This is further evidence that receptor crosslinking is a prerequisite for signal transduction in both the receptor and ligand families. Importantly, the use of receptor-specific monoclonal antibodies or soluble receptors in the form of multimeric Ig fusion proteins has been useful in determining biological function in vitro and in vivo for several family members. Soluble receptor-Ig fusion proteins have been used successfully in the cloning of the cell surface ligands corresponding to the CD40, CD30, CD27, 4-1BB and Fas receptors.

Bone remodeling is the dynamic process whereby skeletal mass and architecture are renewed and maintained. This renewal and maintenance is a balance between bone resorption and bone formation, with the osteoclast and the osteoblast considered the two key participants in the remodeling process. The osteoclast initiates the remodeling cycle by resorbing a cavity in the bone which is subsequently refilled when the osteoblast synthesizes and deposits new bone matrix into the excavation. The activities of osteoclast and osteoblast are regulated by complex interactions between systemic hormones and the local production of growth factors and cytokines at active remodeling sites.

Imbalances in bone remodeling are associated with such conditions as osteoporosis, Paget's disease, and hyperparathyroidism. Osteoporosis, characterized by a decrease in the skeletal mass, is one of the most common diseases of postmenopausal women and is often the cause of debilitating and painful fractures of the spine, hip and wrist.

Bone loss associated with osteoporosis has been arrested by the administration of exogeneous estrogens. To be effective, estrogen therapy must begin within a few years of the onset of menopause, and should continue for 10 to 15 years, according to Thorneycroft (Am. J. Obstet. Gynecol. 160:1306-1310, 1989). While there are several different types of estrogens, 17-.beta.-estradiol is the primary estrogen found naturally occurring in premenopausal women and is often the compound of choice for therapeutic use. At the recommended dose, however, there are significant side effects, the most disturbing being the well-established correlation of estrogen therapy with endometrial and breast cancers. The incidence of carcinoma is both dose-dependent and duration-dependent.

The demonstrated in vivo activities of these TNF ligand family members illustrate the enormous clinical potential of, and need for, other TNF ligands, ligand agonists and antagonists, and TNF receptors. The present invention addresses this need by providing a novel TNF ligand and related compositions and methods.

Within one aspect the invention provides an isolated polypeptide comprising a sequence of amino acid residues from amino acid residue 62 to amino acid residue 81 as shown in SEQ ID NO:2. Within an embodiment, the isolated polypeptide further comprises an amino acid sequence selected from the group consisting of: a) a polypeptide comprising a sequence of amino acids from amino acid residue 20 to amino acid residue 87 as shown in SEQ ID NO:2; a polypeptide that is at least 80% identical to a); b) a polypeptide that is at least 85% identical to a); c) a polypeptide that is at least 90% identical to a); d) a polypeptide that is at least 95% identical to a); and e) a polypeptide that is at least 99% identical to a). Within another embodiment, the isolated polypeptide comprises the amino acid sequence from amino acid 20 to amino acid 87 as shown in SEQ ID NO:2. Within another embodiment, residues 62 through 81 as shown in SEQ ID NO:2 bind a tumor necrosis factor receptor. Within another embodiment, the the polypeptide further comprises a linker region adjacent to the N-terminal of the residues 62 through 81. Within another embodiment, the polypeptide further comprises a transmembrane domain separated from residues 62 through 81 by the linker region. Within another embodiment, the polypeptide further comprises a cytoplasmic region separated from residues 62 through 81 by the transmembrane domain and linker region.

Within another aspect the invention provides, an isolated polypeptide wherein the polypeptide further comprises an amino acid sequence selected from the group consisting of: a) a polypeptide comprising a sequence of amino acids from amino acid residue 1 to amino acid residue 87 as shown in SEQ ID NO:2; b) a polypeptide that is at least 80% identical to a); c) a polypeptide that is at least 85% identical to a); d) a polypeptide that is at least 90% identical to a); e) a polypeptide that is at least 95% identical to a); and f) a polypeptide that is at least 99% identical to a). Within another embodiment, the isolated polypeptide is covalently linked to a moiety selected from the group consisting of affinity tags, toxins, radionucleotides, enzymes and fluorophores. Within another embodiment, the isolated polypeptide further comprising a proteolytic cleavage site between said polypeptide and said moiety.

Within another aspect of the invention is provided a fusion protein consisting essentially of a first portion and a second portion joined by a peptide bond, said first portion consisting essentially of a polypeptide selected from the group consisting of: (a) a polypeptide comprising a sequence of amino acid residues from amino acid residue 20 to amino acid residue 87 of SEQ ID NO:2; (b) a polypeptide having a sequence of amino acid residues that are at least 80% identical to (a); wherein the second portion consisting essentially of a second polypeptide.

Within another aspect the invention provides an antibody that specifically binds to an epitope of a polypeptide of SEQ ID NO:2. Within another embodiment, the antibody is a monoclonal antibody. Within another embodiment, the invention provides a method of producing an antibody comprising the following steps in order: inoculating an animal with a polypeptide selected from the group consisting of: a polypeptide consisting of amino acid residues 68 to 82 as shown in SEQ ID NO:2; a polypeptide consisting of amino acid residues 20 to 87 as shown in SEQ ID NO:2; and a polypeptide consisting of amino acid residues to 87 as shown in SEQ ID NO:2; wherein the polypeptide elicits an immune response in the animal to produce the antibody; and isolating the antibody from the animal; wherein the antibody specifically binds to the amino acid sequence of SEQ ID NO:2 from amino acid number 1 to amino acid number 87.

Within another aspect the invention provides a binding protein that specifically binds to an epitope of a polypeptide of SEQ ID NO:2.

Within another aspect, the invention provides an isolated polynucleotide encoding the amino acid sequence from amino acid residue 62 to amino acid residue 81 as shown in SEQ ID NO:2. Within another embodiment, the isolated polynucleotide encodes a polypeptide comprising an amino acid sequence from amino residue 20 to amino acid residue 87 as shown in SEQ ID NO:2. Within another embodiment, the isolate polynucleotide encodes a polypeptide further comprising amino residue 1 to amino acid residue 87 as shown in SEQ I) NO:2. Within another embodiment, the isolated polynucleotide encodes a polypeptide, wherein the polypeptide further comprises a linker region adjacent to the N-terminal of amino acid residues 62 through 81. Within another embodiment, the isolated polynucleotide encodes a polypeptide that further comprises a transmembrane domain separated from amino acid residues 62 through 81 by the linker region. Within another embodiment, the isolated polynucleotide encodes a polypeptide, wherein the polypeptide further comprises a cytoplasmic region separated from amino acid residues 62 through 81 by a transmembrane domain and a linker region.

Within another aspect the invention provides an expression vector comprising the following operably linked elements: a transcription promoter; a DNA segment encoding the polypeptide from amino acid residue 20 to amino acid residue 87, wherein said polypeptide is a tumor necrosis factor; and a transcription terminator. Within another embodiment, the DNA segment encodes a polypeptide further comprising an affinity tag. Within another embodiment, the invention provides a cultured cell into which has been introduced the expression vector. Within another embodiment, is provided a method of producing a polypeptide comprising: culturing the cell, whereby said cell expresses said polypeptide encoded by said DNA segment; and recovering said expressed polypeptide.

Within another aspect is provided a method for detecting a genetic abnormality in a patient, comprising: obtaining a genetic sample from a patient; incubating the genetic sample with a polynucleotide comprising at least 14 contiguous nucleotides of SEQ ID NO: 1 or the complement of SEQ ID NO: 1, under conditions wherein said polynucleotide will hybridize to complementary polynucleotide sequence, to produce a first reaction product; comparing said first reaction product to a control reaction product, wherein a difference between said first reaction product and said control reaction product is indicative of a genetic abnormality in the patient.

Within another aspect the invention provides a method for the treatment of a mammal having a need for a ZTNF9 polypeptide comprising administering to said mammal a pharmaceutically effective amount of the polypeptide selected from the group consisting of: (a) a polypeptide comprising a sequence of amino acids from amino acid residue 20 to amino acid residue 87 of SEQ ID NO:2; and (b) a polypeptide having a sequence of amino acid residues that are at least 80% identical to (a).

Within another aspect of the invention is provided a method for the treatment of a mammal having a need for an antagonist of a ZTNF9 polypeptide comprising administering to said mammal a pharmaceutically effective amount of an antagonist of a polypeptide selected from the group consisting of: (a) a polypeptide comprising a sequence of amino acids from amino acid residue 20 to amino acid residue 87 of SEQ ID NO:2; (b) a polypeptide having a sequence of amino acid residues that are at least 80% identical to (a).

Prior to setting forth the invention, it may be helpful to an understanding thereof to set forth definitions of certain terms to be used hereinafter:

Affinity tag: is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), Glu-Glu affinity tag (Grussenmeyer et al., Proc. Natl. Acad. Sci. USA 82:7952-4, 1985), substance P, Flag™ peptide (Hopp et al., Biotechnology 6:1204-10, 1988), streptavidin binding peptide, or other antigenic epitope or binding domain. See, in general, Ford et al., Protein Expression and Purification 2: 95-107, 1991. DNAs encoding affinity tags are available from commercial suppliers (e.g., Pharmacia Biotech, Piscataway, N.J.).

Allelic variant : Any of two or more alternative forms of a gene occupying the same chromosomal locus. Allelic variation arises naturally through mutation, and may result in phenotypic polymorphism within populations. Gene mutations can be silent (i.e., no change in the encoded polypeptide), or may encode polypeptides having altered amino acid sequence. The term “allelic variant” is also used herein to denote a protein encoded by an allelic variant of a gene. Also included are the same protein from the same species which differs from a reference amino acid sequence due to allelic variation. Allelic variation refers to naturally occurring differences among individuals in genes encoding a given protein.

Amino-terminal and carboxyl-terminal: are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

Complement/anti-complement pair: Denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of <10⁻⁹ M.

Complements of polynucleotide molecules: Denotes polynucleotide molecules having a complementary base sequence and reverse orientation as compared to a reference sequence. For example, the sequence 5′ ATGCACGGG 3′ is complementary to 5′ CCCGTGCAT 3′.

Contig: denotes a polynucleotide that has a contiguous stretch of identical or complementary sequence to another polynucleotide. Contiguous sequences are said to “overlap” a given stretch of polynucleotide sequence either in their entirety or along a partial stretch of the polynucleotide. For example, representative contigs to the polynucleotide sequence 5′-ATGGCTTAGCTT-3′ are 5′-TAGCTTgagtct-3′ and 3′-gtcgacTACCGA-5′.

Degenerate: As applied to a nucleotide sequence such as a probe or primer, denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

Expression vector: A DNA molecule, linear or circular, that comprises a segment encoding a polypeptide of interest operably linked to additional segments that provide for its transcription. Such additional segments may include promoter and terminator sequences, and optionally one or more origins of replication, one or more selectable markers, an enhancer, a polyadenylation signal, and the like. Expression vectors are generally derived from plasmid or viral DNA, or may contain elements of both.

Isolated: when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

Isolated polypeptide or protein: is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

Operably linked: As applied to nucleotide segments, the term “operably linked” indicates that the segments are arranged so that they function in concert for their intended purposes, e.g., transcription initiates in the promoter and proceeds through the coding segment to the terminator.

Ortholog: denotes a polypeptide or protein obtained from one species that is the functional counterpart of a polypeptide or protein from a different species. Sequence differences among orthologs are the result of speciation.

Paralogs: are distinct but structurally related proteins made by an organism. Paralogs are believed to arise through gene duplication. For example, α-globin, β-globin, and myoglobin are paralogs of each other.

Polynucleotide: Denotes a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired. Such unpaired ends will in general not exceed 20 nt in length.

Polypeptide: as used herein is a polymer of amino acid residues joined by peptide bonds, whether produced naturally or synthetically. Polypeptides of less than about 10 amino acid residues are commonly referred to as “peptides”.

Promoter: Denotes a portion of a gene containing DNA sequences that provide for the binding of RNA polymerase and initiation of transcription. Promoter sequences are commonly, but not always, found in the 5′ non-coding regions of genes.

Protein: is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

Receptor: A cell-associated protein, or a polypeptide subunit of such protein, that binds to a bioactive molecule (the “ligand”) and mediates the effect of the ligand on the cell. Binding of ligand to receptor results in a change in the receptor (and, in some cases, receptor multimerization, i.e., association of identical or different receptor subunits) that causes interactions between the effector domain(s) of the receptor and other molecule(s) in the cell. These interactions in turn lead to alterations in the metabolism of the cell. Metabolic events that are linked to receptor-ligand interactions include gene transcription, phosphorylation, dephosphorylation, cell proliferation, increases in cyclic AMP production, mobilization of cellular calcium, mobilization of membrane lipids, cell adhesion, hydrolysis of inositol lipids and hydrolysis of phospholipids. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

Secretory signal sequence: A DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

Soluble receptor or ligand: A receptor or a ligand polypeptide that is not bound to a cell membrane. Soluble receptors are most commonly ligand-binding receptor polypeptides that lack transmembrane and cytoplasmic domains. Soluble ligands are most commonly receptor-binding polypeptides that lack transmembrane and cytoplasmic domains. Soluble receptors or ligands can comprise additional amino acid residues, such as affinity tags that provide for purification of the polypeptide or provide sites for attachment of the polypeptide to a substrate. Many cell-surface receptors and ligands have naturally occurring, soluble counterparts that are produced by proteolysis or translated from alternatively spliced mRNAs. Receptor and ligand polypeptides are said to be substantially free of transmembrane and intracellular polypeptide segments when they lack sufficient portions of these segments to provide membrane anchoring or signal transduction, respectively.

Splice variant: is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

All references cited herein are incorporated by reference in their entirety.

The present invention is based in part upon the discovery of a novel DNA sequence (SEQ ID NO:1) and corresponding polypeptide sequence (SEQ ID NO:2) which have homology to members of the tumor necrosis factor ligand family, and in particular to RANKL. Analysis of the tissue distribution of the mRNA corresponding to this novel DNA suggests that the ligand is involved in modulating an immune response. The ligand has been designated ZTNF9.

Novel ZTNF9 ligand-encoding polynucleotides and polypeptides of the present invention were initially identified by querying a human genomic database for sequences homologous to RANK ligand—a member of the TNF ligand family. Using this information, a single exon was identified. Using this information, a novel 2,601 bp human cDNA fragment (SEQ ID NO: 1) was identified. Analysis of the DNA (SEQ ID NO: 1) encoding a ZTNF9 polypeptide revealed an open reading frame encoding 87 amino acids (SEQ ID NO: 2) comprising a signal sequence, residue 1 to residue 19 of SEQ ID NO:2, and a receptor-binding domain, residue 20 to 87 of SEQ ID NO:2. Those skilled in the art will recognize that these domain boundaries are approximate, and are based on alignments with known proteins and predictions of protein folding.

The receptor-binding domain of TNF ligands consists of a β-sandwich which contains two sets of 5 anti-parallel beta-strands. ZTNF-9 is a truncated form of a TNF ligand. This truncation will likely cause a simple shift of several beta-strands to form a more compact structure than the typical domain.

Most proteins which are members of the TNF family can be recognized by a conserved 11 amino motif:

[LIVMFY]-X-[LIVMFY]-X-X-X-G-[LIVMFY]-[FY]-[LIVMFY]-[LIVMFY] (SEQ ID NO:4)

wherein amino acid residue 1, 3, 8, 10 and 11 are selected from leucine (L), isoleucine (I), valine (V), methionine (M), phenylalanine (F) or tyrosine (Y); X is any amino acid residue and amino acid residue 9 is selected from phenylalanine (F) or tyrosine (Y). Amino acid residues 1-3 of this motif belong to beta-strand “C” and residues 7-11 belong to beta-strand “D”. This region appears to be critical to the structural integrity of the TNF beta-sandwich structure. A modified form of this motif containing the addition of a Gly to position 1 and the addition of an Arg to position 11 is present in the ZTNF9 -polypeptide from amino acid residue 47 to amino acid residue 57 of SEQ ID NO:2, also shown as SEQ ID NO:6.

Using the crystal structure of AP02L and DR5 (a TNF and TNF receptor in PDB: IDU3), a peptide loop of AP02L is observed to interact with the TNF receptor. Given the homology between RANKL and AP02L, we can assume that the 3D structure of RANKL interacting with RANK would be very similar. Furthermore, the comparison (sequence alignment) between ZTNF9 and RANKL suggests that the homologous peptide loop of ZTNF9 may interact with a TNF receptor in an analogous fashion. This peptide loop comprises a peptide from amino acid 62 of SEQ ID NO:2 to amino acid 81 of SEQ ID NO:2, (i.e., the amino acid sequence:

CSRHRVTSAGLTLQDLQLWC, also shown as SEQ ID NO:4). The loop may form a disulfide bond, which would constrain the peptide and force it into a conformation which may be compatible with binding to a TNF receptor. The specific residues which would interact with the TNF receptor are from residue 70 of SEQ ID NO:2 to residue 78 of SEQ ID NO:2 (i.e., the amino acid sequence: AGLTLQDLQ).

Analysis of the tissue distribution of ZTNF9 can be performed by the Northern blotting technique using Human Multiple Tissue and Master Dot Blots. Such blots are commercially available (Clontech, Palo Alto, Calif.) and can be probed by methods known to one skilled in the art. Also see, for example, Wu W. et al., Methods in Gene Biotechnology, CRC Press LLC, 1997. Additionally, portions of the polynucleotides of the present invention can be identified by querying sequence databases and identifying the tissues from, which the sequences are derived. Portions of the polynucleotides of the present invention have been identified in multiple testis libraries.

ZTNF9 as represented by (SEQ ID NO: 1) was mapped to chromosome 2p25.1 using sequence-tagged-sites (STSs).

The present invention also provides polynucleotide molecules, including DNA and RNA molecules, that encode the ZTNF9 polypeptides disclosed herein. Those skilled in the art will readily recognize that, in view of the degeneracy of the genetic code, considerable sequence variation is possible among these polynucleotide molecules. SEQ ID NO:3 is a degenerate DNA sequence that encompasses all DNAs that encode the ZTNF9 polypeptide of SEQ ID NO:2. Those skilled in the art will recognize that the degenerate sequence of SEQ ID NO:3 also provides all RNA sequences encoding SEQ ID NO:2 by substituting U (uracil) for T (thymine). Thus, ZTNF9 polypeptide-encoding polynucleotides comprising nucleotide 1 to nucleotide 261 of SEQ ID NO:3 and their RNA equivalents are contemplated by the present invention.

Table 1 sets forth the one-letter codes used within SEQ ID NO:3 to denote degenerate nucleotide positions. “Resolutions” are the nucleotides denoted by a code letter. “Complement” indicates the code for the complementary nucleotide(s). For example, the code Y denotes either C (cytosine) or T, and its complement R denotes A (adenine) or G (guanine), A being complementary to T, and G being complementary to C.

TABLE 1

| Nucleotide | Resolution | Nucleotide | Complement |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NO:3, encompassing all possible codons for a given amino acid, are set forth in Table 2.

TABLE 2

| | One | | |
|---|---|---|---|
| Amino | Letter | | Degenerate |
| Acid | Code | Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCAGCCGCGGCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gln | Q | CAA CAG | CAR |
| Hls | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | . | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequence of SEQ ID NO:2. Variant sequences can be readily tested for functionality as described herein.

One of ordinary skill in the art will also appreciate that different species can exhibit “preferential codon usage.” In general, see, Grantham, et al., Nuc. Acids Res. 8:1893-912, 1980; Haas, et al. Curr. Biol. 6:315-24, 1996; Wain-Hobson, et al., Gene 13:355-64, 1981; Grosjean and Fiers, Gene 18:199-209, 1982; Holm, Nuc. Acids Res. 14:3075-87, 1986; Ikemura, J. Mol. Biol. 158:573-97, 1982. As used herein, the term “preferential codon usage” or “preferential codons” is a term of art referring to protein translation codons that are most frequently used in cells of a certain species, thus favoring one or a few representatives of the possible codons encoding each amino acid (See Table 2). For example, the amino acid threonine (Thr) may be encoded by ACA, ACC, ACG, or ACT, but in mammalian cells ACC is the most commonly used codon; in other species, for example, insect cells, yeast, viruses or bacteria, different Thr codons may be preferential. Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequence disclosed in SEQ ID NO:3 serves as a template for optimizing expression of polynucleotides in various cell types and species commonly used in the art and disclosed herein. Sequences containing preferential codons can be tested and optimized for expression in various species, and tested for functionality as disclosed herein.

Within preferred embodiments of the invention, isolated polynucleotides will hybridize to similar sized regions of SEQ ID NO:1, or to a sequence complementary thereto, under stringent conditions. In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typical stringent conditions are those in which the salt concentration is up to about 0.03 M at pH 7 and the temperature is at least about 60° C. As previously noted, the isolated polynucleotides of the present invention include DNA and RNA. Methods for isolating DNA and RNA are well known in the art. It is generally preferred to isolate RNA from testis, although DNA can also be prepared using RNA from other tissues or isolated as genomic DNA. Total RNA can be prepared using guanidine HCl extraction followed by isolation by centrifugation in a CsCl gradient (Chirgwin et al., Biochemistry 18:52-94, 1979). Poly (A)⁺ RNA is prepared from total RNA using the method of Aviv and Leder (Proc. Natl. Acad. Sci. USA 69:1408-12, 1972). Complementary DNA (cDNA) is prepared from poly(A)⁺ RNA using known methods. Polynucleotides encoding ZTNF9 polypeptides are then identified and isolated by, for example, hybridization or PCR.

Those skilled in the art will recognize that the sequence disclosed in SEQ ID NO:1 represents a single allele of the human ZTNF9 gene, and that allelic variation and alternative splicing are expected to exist. Allelic variants of the DNA sequence shown in SEQ ID NO: 1, including those containing silent mutations and those in which mutations result in amino acid sequence changes, are within the scope of the present invention, as are proteins which are allelic variants of SEQ ID NO:2. cDNAs generated from alternatively spliced mRNAs, which retain the properties of the ZTNF9 polypeptide are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art.

The present invention further provides counterpart ligands and polynucleotides from other species (“species orthologs”). These species include, but are not limited to mammalian, avian, amphibian, reptile, fish, insect and other vertebrate and invertebrate species. Of particular interest are ZTNF9 ligand polypeptides from other mammalian species, including murine, porcine, ovine, bovine, canine, feline, equine, and other primate ligands. Species orthologs of human ZTNF9 can be cloned using information and compositions provided by the present invention in combination with conventional cloning techniques. For example, a cDNA can be cloned using mRNA obtained from a tissue or cell type that expresses the ligand. Suitable sources of mRNA can be identified by probing Northern blots with probes designed from the sequences disclosed herein. A library is then prepared from mRNA of a positive tissue or cell line. A ZTNF9-encoding cDNA can then be isolated by a variety of methods, such as by probing with a complete or partial human cDNA or with one or more sets of degenerate probes based on the disclosed sequence. A cDNA can also be cloned using the polymerase chain reaction (PCR) (Mullis, U.S. Pat. No. 4,683,202), using primers designed from the sequences disclosed herein. Within an additional method, the cDNA library can be used to transform or transfect host cells, and expression of the cDNA of interest can be detected with an antibody to ZTNF9. Similar techniques can also be applied to the isolation of genomic clones.

Alternate species polypeptides of ZTNF9 may have importance therapeutically. It has been demonstrated that in some cases use of a non-native protein, i.e., protein from a different species, can be more potent than the native protein.

For example, salmon calcitonin has been shown to be considerably more effective in arresting bone resorption than human forms of calcitonin. There are several hypotheses as to why salmon calcitonin is more potent than human calcitonin in treatment of osteoporosis. These hypotheses include: 1) salmon calcitonin is more resistant to degradation; 2) salmon calcitonin has a lower metabolic clearance rate (MCR); and 3) salmon calcitonin may have a slightly different conformation, resulting in a higher affinity for bone receptor sites. Another example is found in the β-endorphin family (Ho et al., Int. J. Peptide Protein Res. 29:521-4, 1987). Studies have demonstrated that the peripheral opioid activity of camel, horse, turkey and ostrich β-endorphins is greater than that of human β-endorphins when isolated guinea pig ileum was electrostimulated and contractions were measured. Vas deferens from rat, mouse and rabbit were assayed as well. In the rat vas deferens model, camel and horse β-endorphins showed the highest relative potency. Synthesized rat relaxin was as active as human and porcine relaxin in the mouse symphysis pubis assay (Bullesbach and Schwabe, Eur. J. Biochem. 241:533-7, 1996). Thus, the mouse ZTNF9 molecules of the present invention may have higher potency than the human endogenous molecule in human cells, tissues and recipients.

The present invention also provides isolated ligand polypeptides that are substantially homologous to the ligand polypeptide of SEQ ID NO:2 and its species orthologs. By “isolated” is meant a protein or polypeptide that is found in a condition other than its native environment, such as apart from blood and animal tissue. In a preferred form, the isolated protein or polypeptide is substantially free of other proteins or polypeptides, particularly other proteins or polypeptides of animal origin. It is preferred to provide the proteins or polypeptides in a highly purified form, i.e. greater than 95% pure, more preferably greater than 99% pure. The term “substantially homologous” is used herein to denote proteins or polypeptides having 50%, preferably 60%, more preferably at least 80%, sequence identity to the sequence shown in SEQ ID NO:2 or its species orthologs. Such proteins or polypeptides will more preferably be at least 90% identical, and most preferably 95% or more identical to SEQ ID NO:2 or its species orthologs or paralogs. Percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown in Table 3 (amino acids are indicated by the standard one-letter codes). The percent identity is then calculated as:

$$\frac{Total number of identical matches}{\begin{matrix}\begin{matrix}[ length of the longer sequence plus the \\ number of gaps introduced into the longer \end{matrix} \\ sequence in order to align the two sequences ]\end{matrix}} \times 100$$

Sequence identity of polynucleotide molecules is determined by similar methods using a ratio as disclosed above.

TABLE 3

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Substantially homologous proteins and polypeptides are characterized as having one or more amino acid substitutions, deletions or additions. These changes are preferably of a minor nature, that is conservative amino acid substitutions (see Table 4) and other substitutions that do not significantly affect the folding or activity of the protein or polypeptide; small deletions, typically of one to about 30 armino acids; and small amino- or carboxyl-terminal extensions, such as an amino-terminal methionine residue, a small linker peptide of up to about 20-25 residues, or an affinity tag. The present invention thus includes polypeptides of from 184 to 1000 amino acid residues that comprise a sequence that is at least 60%, preferably at least 80%, and more preferably 90% and even more preferably 95% or more identical to the corresponding region of SEQ ID NO:2. Polypeptides comprising affinity tags can further comprise a proteolytic cleavage site between the ZTNF9 polypeptide and the affinity tag. Preferred such sites include thrombin cleavage sites and factor Xa cleavage sites.

TABLE 4
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of ZTNF9 polypeptides of the present invention. A limited number of non-conservative amino acids, amino acids that are not encoded by the genetic code, and unnatural amino acids may be substituted for ZTNF9 polypeptide amino acid residues. The proteins of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for ZTNF9 amino acid residues.

Essential amino acids in the ZTNF9 polypeptides of the present invention can be identified according to procedures known in the art, such as site-directed mutagenesis or alanine-scanning mutagenesis (Cunningham and Wells, Science 244: 1081-5, 1989). Sites of biological interaction, such as ZTNF9 polypeptide-cysteine proteinase inhibitor-enzyme interaction, can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related cystatin family members.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed (Derbyshire et al., Gene 46:145, 1986; Ner et al., DNA 7:127, 1988).

Variants of the disclosed ZTNF9 DNA and polypeptide sequences can be generated through DNA shuffling as disclosed by Stemmer, Nature 370:389-91, 1994, Stemmer, Proc. Natl. Acad. Sci. USA 91:10747-51, t994 and WIPO Publication WO 97/20078. Briefly, variant DNAs are generated by in vitro homologous recombination by random fragmentation of a parent DNA followed by reassembly using PCR, resulting in randomly introduced point mutations. This technique can be modified by using a family of parent DNAs, such as allelic variants or DNAs from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Mutagenesis methods as disclosed above can be combined with high-throughput screening methods to detect activity of cloned, mutagenized ligands. Mutagenized DNA molecules that encode active ligands or portions thereof (e.g., receptor-binding fragments) can be recovered from the host cells and rapidly sequenced using modern equipment. These methods allow the rapid determination of the importance of individual amino acid residues in a polypeptide of interest, and can be applied to polypeptides of unknown structure.

Using the methods discussed above, one of ordinary skill in the art can identify and/or prepare a variety of polypeptides that are substantially homologous to residues 20 to 87 of SEQ ID NO: 2 or allelic variants thereof and retain the receptor-binding properties of the wild-type protein. Such polypeptides may include additional amino acids from the transmembrane domain, linker and/or cytoplasmic domain;

affinity tags; and the like. Such polypeptides may also include additional polypeptide segments as generally disclosed above.

The ligand polypeptides of the present invention, including full-length ligand polypeptides, ligand fragments (e.g., receptor-binding fragments), and fusion polypeptides, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, Second Edition, Cold Spring Harbor, N.Y., 1989; and Ausubel et al., eds., Current Protocols in Molecular Biology, John Wiley and Sons, Inc., N.Y., 1987.

For any ZTNF9 polypeptide, including variants and fusion proteins, one of ordinary skill in the art can readily generate a fully degenerate polynucleotide sequence encoding that variant using the information set forth in Tables 1 and 2 above.

In general, a DNA sequence encoding a ZTNF9 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a ZTNF9 polypeptide into the secretory pathway of a host cell, a secretory signal sequence (also known as a signal sequence, leader sequence, prepro sequence or pre sequence) is provided in the expression vector. The secretory signal sequence may be derived from another secreted protein (e.g., t-PA) or synthesized de novo. The secretory signal sequence is joined to the ZTNF9 DNA sequence in the correct reading frame and positioned to direct the newly synthesized polypeptide into the secretory pathway of the host cell. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830).

Since multimeric complexes of the TNF ligand and TNF receptor families are known to be biologically active, it may be useful to prepare fusion proteins of ZTNF9 with another TNF ligand. One such ligand, for example, is RANK-L. The fusion protein can be prepared with the ZTNF9 polynucleotide sequence, or a portion thereof, at the amino terminal followed by the carboxyl terminal of RANK-L. Similarly, ztnf9 polypeptides, or fragments thereof, can be used as an agonist of RANKL activity by binding the RANK receptor and stimulating osteoclast activity. (See Li, J. et al., P.N.A.S. 1566-1571, 2000.) Alternatively, these polypeptides can be used as an inhibitor of RANK-L activity by binding the RANK receptor, but failing to result in an intracellular signal.

As discussed above, it is likely that ZTNF9 polypeptides will form a trimer to facilitate receptor binding. Of note, however, it may not be necessary for TNF receptor polypeptides to form a trimeric complex. Bazzoni (Bazzoni, F. et al., P.N.A.S.92: 5376-5380, 1995) have shown that for some TNF receptors, dimerization (rather than trimerization or higher-order multimerization) was sufficient. Thus, ZTNF9 polypeptides may be useful as dimers, timers, or a combination thereof.

Cultured mammalian cells are suitable hosts within the present invention. Methods for introducing exogenous DNA into mammalian host cells include calcium phosphate-mediated transfection (Wigler et al., Cell 14:725, 1978;

Corsaro and Pearson, Somatic Cell Genetics 7:603, 1981; Graham and Van der Eb, Virology 52:456, 1973), electroporation (Neumann et al., EMBO J. 1:841-45, 1982), DEAE-dextran mediated transfection (Ausubel et al., ibid), and liposome-mediated transfection (Hawley-Nelson et al., Focus 15:73, 1993; Ciccarone et al., Focus 15:80, 1993). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g., CHO-KI; ATCC No. CCL 61) cell lines. Additional suitable cell lines are known in the art and available from public depositories such as the American Type Culture Collection, Rockville, Md. In general, strong transcription promoters are preferred, such as promoters from SV-40 or cytomegalovirus. See, e.g., U.S. Pat. No. 4,956,288. Other suitable promoters include those from metallothionein genes (U.S. Patent Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems may also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g., hygromycin resistance, multi-drug resistance, puromycin acetyltransferase) can also be used. Alternative markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

Other higher eukaryotic cells can also be used as hosts, including plant cells, insect cells and avian cells. The use of Agrobacterium rhizogenes as a vector for expressing genes in plant cells has been reviewed by Sinkar et al., J. Biosci.(Bangalore) 11:47-58, 1987. Transformation of insect cells and production of foreign polypeptides therein is disclosed by Guarino et al., U.S. Pat. No. 5,162,222 and WIPO publication WO 94/06463. Insect cells can be infected with recombinant baculovirus, commonly derived from Autographa californica nuclear polyhedrosis virus (AcNPV). DNA encoding the ZTNF9 polypeptide is inserted into the baculoviral genome in place of the AcNPV polyhedrin gene coding sequence by one of two methods. The first is the traditional method of homologous DNA recombination between wild-type AcNPV and a transfer vector containing the ZTNF9 flanked by AcNPV sequences. Suitable insect cells, e.g. SF9 cells, are infected with wild-type AcNPV and transfected with a transfer vector comprising a ZTNF9 polynucleotide operably linked to an AcNPV polyhedrin gene promoter, terminator, and flanking sequences. See, King and Possee, The Baculovirus Expression System: A Laboratory Guide, London, Chapman & Hall; O'Reilly et al., Baculovirus Expression Vectors: A Laboratory Manual, New York, Oxford University Press., 1994; and, Richardson (Ed.), Baculovirus Expression Protocols. Methods in Molecular Biology, Totowa, N.J., Humana Press, 1995. Natural recombination within an insect cell will result in a recombinant baculovirus which contains ZTNF9 driven by the polyhedrin promoter. Recombinant viral stocks are made by methods commonly used in the art.

The second method of making recombinant baculovirus utilizes a transposon-based system described by Luckow et al. (J. Virol. 67:4566-79, 1993). This system is sold in the Bac-to-Bac kit (Life Technologies, Rockville, Md.). This system utilizes a transfer vector, pFastBac1™ (Life Technologies) containing a Tn7 transposon to move the DNA encoding the ZTNF9 polypeptide into a baculovirus genome maintained in E. coli as a large plasmid called a “bacmid.” The pFastBac1™ transfer vector utilizes the AcNPV polyhedrin promoter to drive the expression of the gene of interest, in this case ZTNF9. However, pFastBac1™ can be modified to a considerable degree. The polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins. See, Hill-Perkins and Possee, J. Gen. Virol. 71:971-6, 1990; Bonning et al., J. Gen. Virol. 75:1551-6, 1994; and, Chazenbalk and Rapoport, J. Biol. Chem. 270:1543-9, 1995. In such transfer vector constructs, a short or long version of the basic protein promoter can be used. Moreover, transfer vectors can be constructed which replace the native ZTNF9 secretory signal sequences with secretory signal sequences derived from insect proteins. For example, a secretory signal sequence from Ecdysteroid Glucosyltransferase (EGT), honey bee Melittin (Invitrogen, Carlsbad, Calif.), or baculovirus gp67 (PharMingen, San Diego,Calif.) can be used in constructs to replace the native ZTNF9 secretory signal sequence. In addition, transfer vectors can include an in-frame fusion with DNA encoding an epitope tag at the C- or N-terminus of the expressed ZTNF9 polypeptide, for example, a Glu-Glu epitope tag (Grussenmeyer et al., Proc. Natl. Acad. Sci. 82:7952-4, 1985) or FLAG tag. Using a technique known in the art, a transfer vector containing ZTNF9 is transformed into E. coli, and screened for bacmids which contain an interrupted lacZ gene indicative of recombinant baculovirus. The bacmid DNA containing the recombinant baculovirus genome is isolated, using common techniques, and used to transfect Spodoptera frugiperda cells, e.g. Sf9 cells. Recombinant virus that expresses ZTNF9 is subsequently produced. Recombinant viral stocks are made by methods commonly used the art.

The recombinant virus is used to infect host cells, typically a cell line derived from the fall armyworm, Spodoptera frugiperda. See, in general, Glick and Pasternak, Molecular Biotechnology: Principles and Applications of Recombinant DNA, ASM Press, Washington, D.C., 1994. Another suitable cell line is the High FiveO™ cell line (Invitrogen) derived from Trichoplusia ni (U.S. Patent #5,300,435). Commercially available serum-free media are used to grow and maintain the cells. Suitable media are Sf900II™ (Life Technologies) or ESF 921™ (Expression Systems) for the Sf9 cells; and Ex-cellO405™ (JRH Biosciences, Lenexa, Kans.) or Express FiveO™ (Life Technologies) for the T. ni cells. The cells are grown up from an inoculation density of approximately 2-5×105 cells to a density of 1-2×10⁶ cells at which time a recombinant viral stock is added at a multiplicity of infection (MOI) of 0.1 to 10, more typically near 3. The recombinant virus-infected cells typically produce the recombinant ZTNF9 polypeptide at 12-72 hours post-infection and secrete it with varying efficiency into the medium. The culture is usually harvested 48 hours post-infection. Centrifugation is used to separate the cells from the medium (supernatant). The supernatant containing the ZTNF9 polypeptide is filtered through micropore filters, usually 0.45 μm pore size. Procedures used are generally described in available laboratory manuals (King and Possee, ibid.; O'Reilly et al., ibid.; Richardson, ibid.). Subsequent purification of the ZTNF9 polypeptide from the supernatant can be achieved using methods described herein.

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in S. cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Patents Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, P. pastoris, P. methanolica, P. guillermondii and Candida maltosa are known in the art. See, for example, Gleeson et al., J. Gen. Microbiol. 132:3459-65, 1986 and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

The use of Pichia methanolica as host for the production of recombinant proteins is disclosed in WIPO Publications WO 97/17450, WO 97/17451, WO 98/02536, and WO 98/02565. DNA molecules for use in transforming P. methanolica will commonly be prepared as double-stranded, circular plasmids, which are preferably linearized prior to transformation. For polypeptide production in P. methanolica, it is preferred that the promoter and terminator in the plasmid be that of a P. methanolica gene, such as a P. methanolica alcohol utilization gene (AUG1 or AUG2). Other useful promoters include those of the dihydroxyacetone synthase (DHAS), formate dehydrogenase (FMD), and catalase (CAT) genes. To facilitate integration of the DNA into the host chromosome, it is preferred to have the entire expression segment of the plasmid flanked at both ends by host DNA sequences. A preferred selectable marker for use in Pichia methanolica is a P. methanolica ADE2 gene, which encodes phosphoribosyl-5-aminoimidazole carboxylase (AIRC; EC 4.1.1.21), which allows ade2 host cells to grow in the absence of adenine. For large-scale, industrial processes where it is desirable to minimize the use of methanol, it is preferred to use host cells in which both methanol utilization genes (AUG1 and AUG2) are deleted. For production of secreted proteins, host cells deficient in vacuolar protease genes (PEP4 and PRB1) are preferred. Electroporation is used to facilitate the introduction of a plasmid containing DNA encoding a polypeptide of interest into P. methanolica cells. It is preferred to transform P. methanolica cells by electroporation using an exponentially decaying, pulsed electric field having a field strength of from 2.5 to 4.5 kV/cm, preferably about 3.75 kV/cm, and a time constant (τ) of from 1 to 40 milliseconds, most preferably about 20 milliseconds.

Prokaryotic host cells, including strains of the bacteria Escherichia coli, Bacillus and other genera are also useful host cells within the present invention. Techniques for transforming these hosts and expressing foreign DNA sequences cloned therein are well known in the art (see, e.g., Sambrook et al., ibid.). When expressing a ZTNF9 polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell. P. methanolica cells are cultured in a medium comprising adequate sources of carbon, nitrogen and trace nutrients at a temperature of about 25° C. to 35° C. Liquid cultures are provided with sufficient aeration by conventional means, such as shaking of small flasks or sparging of fermentors. A preferred culture medium for P. methanolica is YEPD (2% D-glucose, 2% Bacto™ Peptone (Difco Laboratories, Detroit, Mich.), 1% Bacto™ yeast extract (Difco Laboratories), 0.004% adenine and 0.006% L-leucine).

Expressed recombinant ZTNF9 polypeptides (or chimeric ZTNF9 polypeptides) can be purified using fractionation and/or conventional purification methods and media. Ammonium sulfate precipitation and acid or chaotrope extraction may be used for fractionation of samples. Exemplary purification steps may include hydroxyapatite, size exclusion, FPLC and reverse-phase high performance liquid chromatography. Suitable anion exchange media include derivatized dextrans, agarose, cellulose, polyacrylamide, specialty silicas, and the like. PEI, DEAE, QAE and Q derivatives are preferred, with DEAE Fast-Flow Sepharose (Pharmacia, Piscataway, N.J.) being particularly preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties.

Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Methods for binding receptor polypeptides to support media are well known in the art. Selection of a particular method is a matter of routine design and is determined in part by the properties of the chosen support. See, for example, Affinity Chromatography: Principles & Methods, Pharmacia LKB Biotechnology, Uppsala, Sweden, 1988.

The polypeptides of the present invention can be isolated by exploitation of their physical properties. For example, immobilized metal ion adsorption (IMAC) chromatography can be used to purify histidine-rich proteins, including those having His-tags. Briefly, a gel is first charged with divalent metal ions to form a chelate (E. Sulkowski, Trends in Biochem. 3:1-7, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (Methods in Enzymol., Vol. 182, “Guide to Protein Purification”, M. Deutscher, (ed.), Acad. Press, San Diego, 1990, pp.529-39). Within additional embodiments of the invention, a fusion of the polypeptide of interest and an affinity tag (e.g., Glu-Glu, FLAG, maltose-binding protein, an immunoglobulin domain) may be constructed to facilitate purification.

Protein refolding (and optionally reoxidation) procedures may be advantageously used. It is preferred to purify the protein to >80% purity, more preferably to >90% purity, even more preferably >95%, and particularly preferred is a pharmaceutically pure state, that is greater than 99.9% pure with respect to contaminating macromolecules, particularly other proteins and nucleic acids, and free of infectious and pyrogenic agents. Preferably, a purified protein is substantially free of other proteins, particularly other proteins of animal origin.

ZTNF9 polypeptides or fragments thereof may also be prepared through chemical synthesis. ZTNF9 polypeptides may be monomers or multimers; glycosylated or non-glycosylated; pegylated or non-pegylated; and may or may not include an initial methionine amino acid residue.

The invention also provides soluble ZTNF9 ligands. Preferably the soluble ligand comprises amino acid residues 62 to 81 of SEQ ID NO:2 or the corresponding region of a non-human ligand. One such preferred soluble ZTNF9 ligand comprises amino acid residues 20-87 of SEQ ID NO:2, nucleotides 1203 to 1407 of SEQ ID NO:1. Such soluble polypeptides can be used to form fusion proteins with human Ig, as His-tagged proteins or as N- or C-terminal FLAG™-tagged (Hopp et al., Biotechnology 6:1204-10, 1988) or Glu-Glu tagged proteins. It is preferred that the extracellular receptor-binding domain polypeptides be prepared in a form substantially free of transmembrane and intracellular polypeptide segments. For example, the N-terminus of the receptor-binding domain may be at amino acid residue 20 of SEQ ID NO:2 or at the corresponding region of an allelic variant or a non-human ligand. To direct the export of the soluble ligand from the host cell, the truncated ligand DNA is linked to a second DNA segment encoding a secretory peptide, such as a t-PA secretory peptide. To facilitate purification of the secreted soluble ligand, a C-terminal extension, such as a poly-histidine tag, substance P, Flag™ peptide (Hopp et al., ibid; available from Eastman Kodak Co., New Haven, Conn.) or another polypeptide or protein for which an antibody or other specific binding agent is available, can be fused to the soluble ligand polypeptide at either the N or C terminus.

In an alternative approach, an extracellular receptor-binding domain can be expressed as a fusion with immunoglobulin heavy chain constant regions, typically an F_{c} fragment, which contains two constant region domains and a hinge region, but lacks the variable region. Such fusions are typically secreted as multimeric molecules, wherein the Fc portions are disulfide bonded to each other and two ligand polypeptides are arrayed in close proximity to each other. Fusions of this type can be used to affinity purify the cognate receptor from solution, as an in vitro assay tool, and to block signals in vitro by specifically titrating out or blocking endogenous ligand. To purify soluble receptor, a ZTNF9-Ig fusion protein (chimera) is added to a sample containing the soluble receptor under conditions that facilitate receptor-ligand binding (typically near-physiological temperature, pH, and ionic strength). The chimera-receptor complex is then separated from the mixture using protein A, which is immobilized on a solid support (e.g., insoluble resin beads). The receptor is then eluted using conventional chemical techniques, such as with a salt or pH gradient. In the alternative, the chimera itself can be bound to a solid support, with binding and elution carried out as above. Collected fractions can be re-fractionated until the desired level of purity is reached. For use in assays, the chimeras are bound to a support via the Fc region and used in an ELISA format. Conversely, soluble TNF receptor-Ig fusion proteins may be made using TNF receptors for which a ligand has not been identified. Soluble ZTNF9 is then mixed with a receptor fusion protein and binding is assayed as described above. The chimeras may be used in vivo as an anti-inflammatory, in the inhibition of autoimmune processes, for inhibition of antigen in humoral and cellular immunity and for immunosuppression in graft and organ transplants. The chimeras may also be used to stimulate lymphocyte development, such as during bone marrow transplantation and as therapy for some cancers.

An assay system that uses a ligand-binding receptor (or an antibody, one member of a complement/anti-complement pair) or a binding fragment thereof, and a commercially available biosensor instrument (BIAcore™, Pharmacia Biosensor, Piscataway, N.J.) may be advantageously employed. Such receptor, antibody, member of a complement/anti-complement pair or fragment is immobilized onto the surface of a receptor chip. Use of this instrument is disclosed by Karlsson, J. Immunol. Methods 145:229-40, 1991 and Cunningham and Wells, J. Mol. Biol. 234:554-63, 1993. A receptor, antibody, member or fragment is covalently attached, using amine or sulfhydryl chemistry, to dextran fibers that are attached to gold film within the flow cell. A test sample is passed through the cell. If a ligand, epitope, or opposite member of the complement/anti-complement pair is present in the sample, it will bind to the immobilized receptor, antibody or member, respectively, causing a change in the refractive index of the medium, which is detected as a change in surface plasmon resonance of the gold film. This system allows the determination of on- and off-rates, from which binding affinity can be calculated, and assessment of stoichiometry of binding.

ZTNF9 polynucleotides and/or polypeptides may be useful for regulating the proliferation and stimulation of a wide variety of TNF receptor-bearing cells, such as T cells, lymphocytes, peripheral blood mononuclear cells, polymorphonuclear leukocytes, fibroblasts, hematopoietic cells and a variety of cells in testis tissue. Other tumor necrosis factors, such as gp39 and TNFβ also stimulate B cell proliferation. ZTNF9 polypeptides will also find use in mediating metabolic or physiological processes in vivo. Proliferation and differentiation can be measured in vitro using cultured cells. Bioassays and ELISAs are available to measure cellular response to ZTNF9, in particular are those which measure changes in cytokine production as a measure of cellular response (see for example, Current Protocols in Immunology ed. John E. Coligan et al., NIH, 1996). Assays to measure other cellular responses, including antibody isotype, monocyte activation, NK cell formation, antigen presenting cell function, apoptosis.

A variety of assays are also available to measure bone formation and resorption. These assays measure, for example, serum calcium levels, osteoclast size and number, osteoblast size and number, ostenopenia induced by estrogen deficiency, cancellous bone volumes of the distal femur (mouse), cartilaginous growth plates, and chondrocyte formation and differentiation. The ztnf9 polypeptides of the present invention can be measured in any of these assay, as well as additional assays dislcosed herein, and assays that are readily known to one of skill in the art.

In a preferred embodiment, the cell activation is determined by measuring proliferation using ³H-thymidine uptake (Crowley et al., J. Immunol. Meth. 133:55-66, 1990). Alternatively, cell activation can be measured by the production of cytokines, such as IL-2, or by determining the presence of cell-specific activation markers. Cytokine production can be assayed by testing the ability of the ZTNF9 and cell culture supernatant to stimulate growth of cytokine-dependent cells. Cell specific activation markers may be detected using antibodies specific for such markers.

In vitro and in vivo response to ZTNF9 can also be measured using cultured cells or by administering molecules of the claimed invention to the appropriate animal model. For instance, ZTNF9 transfected expression host cells may be embedded in an alginate environment and injected (implanted) into recipient animals. Alginate-poly-L-lysine microencapsulation, permselective membrane encapsulation and diffusion chambers have been described as a means to entrap transfected mammalian cells or primary mammalian cells. These types of non-immunogenic “encapsulations” or microenvironments permit the transfer of nutrients into the microenvironment, and also permit the diffusion of proteins and other macromolecules secreted or released by the captured cells across the environmental barrier to the recipient animal. Most importantly, the capsules or microenvironments mask and shield the foreign, embedded cells from the recipient animal's immune response. Such microenvironments can extend the life of the injected cells from a few hours or days (naked cells) to several weeks (embedded cells).

Alginate threads provide a simple and quick means for generating embedded cells. The materials needed to generate the alginate threads are readily available and relatively inexpensive. Once made, the alginate threads are relatively strong and durable, both in vitro and, based on data obtained using the threads, in vivo. The alginate threads are easily manipulable and the methodology is scalable for preparation of numerous threads. In an exemplary procedure, 3% alginate is prepared in sterile H₂O, and sterile filtered. Just prior to preparation of alginate threads, the alginate solution is again filtered. An approximately 50% cell suspension (containing about 5×10 to about 5×10⁷ cells/ml) is mixed with the 3% alginate solution. One ml of the alginate/cell suspension is extruded into a 100 mM sterile filtered CaCl₂ solution over a time period of ˜15 min, forming a “thread”. The extruded thread is then transferred into a solution of 50 mM CaCl₂, and then into a solution of 25 mM CaCl₂. The thread is then rinsed with deionized water before coating the thread by incubating in a 0.01% solution of poly-L-lysine. Finally, the thread is rinsed with Lactated Ringer's Solution and drawn from solution into a syringe barrel (without needle attached). A large bore needle is then attached to the syringe, and the thread is intraperitoneally injected into a recipient in a minimal volume of the Lactated Ringer's Solution.

An alternative in vivo approach for assaying proteins of the present invention involves viral delivery systems. Exemplary viruses for this purpose include adenovirus, herpesvirus, vaccinia virus and adeno-associated virus (AAV). Adenovirus, a double-stranded DNA virus, is currently the best studied gene transfer vector for delivery of heterologous nucleic acid (for a review, see Becker et al., Meth. Cell Biol. 43:161-89, 1994; and Douglas and Curiel, Science & Medicine 4:44-53, 1997). The adenovirus system offers several advantages: adenovirus can (i) accommodate relatively large DNA inserts; (ii) be grown to high-titer; (iii) infect a broad range of mammalian cell types; and (iv) be used with a large number of available vectors containing different promoters. Also, because adenoviruses are stable in the bloodstream, they can be administered by intravenous injection. Some disadvantages (especially for gene therapy) associated with adenovirus gene delivery include: (i) very low efficiency integration into the host genome; (ii) existence in primarily episomal form; and (iii) the host immune response to the administered virus, precluding readministration of the adenoviral vector.

By deleting portions of the adenovirus genome, larger inserts (up to 7 kb) of heterologous DNA can be accommodated. These inserts can be incorporated into the viral DNA by direct ligation or by homologous recombination with a co-transfected plasmid. In an exemplary system, the essential E1 gene has been deleted from the viral vector, and the virus will not replicate unless the E1 gene is provided by the host cell (the human 293 cell line is exemplary). When intravenously administered to intact animals, adenovirus primarily targets the liver. If the adenoviral delivery system has an E1 gene deletion, the virus cannot replicate in the host cells. However, the host's tissue (e.g., liver) will express and process (and, if a signal sequence is present, secrete) the heterologous protein. Secreted proteins will enter the circulation in the highly vascularized liver, and effects on the infected animal can be determined.

The adenovirus system can also be used for protein production in vitro. By culturing adenovirus-infected non-293 cells under conditions where the cells are not rapidly dividing, the cells can produce proteins for extended periods of time. For instance, BHK cells are grown to confluence in cell factories, then exposed to the adenoviral vector encoding the secreted protein of interest. The cells are then grown under serum-free conditions, which allows infected cells to survive for several weeks without significant cell division. Alternatively, adenovirus vector infected 293S cells can be grown in suspension culture at relatively high cell density to produce significant amounts of protein (see Garnier et al., Cytotechnol. 15:145-55, 1994). With either protocol, an expressed, secreted heterologous protein can be repeatedly isolated from the cell culture supernatant. Within the infected 293S cell production protocol, non-secreted proteins may also be effectively obtained.

Well established animal models are available to test in vivo efficacy of ZTNF9 polypeptides for certain disease states. In particular, ZTNF9 polypeptides can be tested in vivo in a number of animal models of autoimmune disease, such as the NOD mice, a spontaneous model system for insulin-dependent diabetes mellitus (IDDM), to study induction of non-responsiveness in the animal model. Administration of ZTNF9 polypeptides prior to or after onset of disease can be monitored by assay of urine glucose levels in the NOD mouse. Alternatively, induced models of autoimmune disease, such as experimental allergic encephalitis (EAE), can be administered ZTNF9 polypeptides. Administration in a preventive or intervention mode can be followed by monitoring the clinical symptoms of EAE.

ZTNF9 polypeptides can also be used to prepare antibodies that specifically bind to ZTNF9 epitopes, peptides or polypeptides. Methods for preparing polyclonal and monoclonal antibodies are well known in the art (see, for example, Sambrook et al., Molecular Cloning: A Laboratory Manual, Second Edition, Cold Spring Harbor, N.Y., 1989; and Hurrell, J. G. R., Ed., Monoclonal Hybridoma Antibodies: Techniques and Applications, CRC Press, Inc., Boca Raton, Fla., 1982). As would be evident to one of ordinary skill in the art, polyclonal antibodies can be generated from a variety of warm-blooded animals, such as horses, cows, goats, sheep, dogs, chickens, rabbits, mice, and rats.

The immunogenicity of a ZTNF9 polypeptide may be increased through the use of an adjuvant, such as alum (aluminum hydroxide) or Freund's complete or incomplete adjuvant. Polypeptides useful for immunization also include fusion polypeptides, such as fusions of ZTNF9 or a portion thereof with an immunoglobulin polypeptide or with maltose binding protein. The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

As used herein, the term “antibodies” includes polyclonal antibodies, affinity-purified polyclonal antibodies, monoclonal antibodies, and antigen-binding fragments thereof, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. Non-human antibodies may be humanized by grafting only non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced. Humanized monoclonal antibodies directed against ZTNF9 polypeptides could be used as a protein therapeutic, in particular for use as an immunotherapy. Alternative techniques for generating or selecting antibodies useful herein include in vitro exposure of testis tissue to ZTNF9 protein or peptide, and selection of antibody display libraries in phage or similar vectors (for instance, through use of immobilized or labeled ZTNF9 protein or peptide).

Antibodies are defined to be specifically binding if they bind to a ZTNF9 polypeptide with a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art (for example, by Scatchard analysis).

A variety of assays known to those skilled in the art can be utilized to detect antibodies which specifically bind to ZTNF9 proteins or peptides. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radioimmunoassay, radioimmuno-precipitation, ELISA, dot blot or Western blot assay, inhibition or competition assay, and sandwich assay. In addition, antibodies can be screened for binding to wild-type versus mutant ZTNF9 protein or peptide.

Antibodies to ZTNF9 may be used for immunohistochemical tagging of cells that express human ZTNF9, for example, to use in a diagnostic assays; for isolating ZTNF9 by affinity purification; for screening expression libraries; for generating anti-idiotypic antibodies; and as neutralizing antibodies or as antagonists to block ZTNF9 in vitro and in vivo. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates. Antibodies herein may also be directly or indirectly conjugated to drugs, toxins, radionuclides and the like, and these conjugates used for in vivo diagnostic or therapeutic applications.

Antibodies to soluble ZTNF9 polypeptides can also be prepared. A preferred soluble ZTNF9 polypeptide comprises the sequence of SEQ ID NO:2 from amino acid residue 20 to amino acid residue 87, or to the amino acid loop, i.e., amino acid residue 62 to amino acid 81 of SEQ ID NO:2, or to an interaction region from amino acid 70 to amino acid 78 of SEQ ID NO:2. Preferably such soluble polypeptides are His, Glu-Glu or FLAG tagged. Alternatively such polypeptides form a fusion protein with human Ig. In particular, antiserum containing anti-polypeptide antibodies directed to His-, Glu-Glu- or FLAG-tagged soluble ZTNF9 can be used in analysis of tissue distribution of ZTNF9 or receptors that bind ZTNF9 by immunohistochemistry on human or primate tissue. These soluble ZTNF9 polypeptides can also be used to immunize mice in order to produce monoclonal antibodies to a soluble human ZTNF9 polypeptide. Monoclonal antibodies to a soluble human ZTNF9 polypeptide can be used to analyze hematopoietic cell distribution using methods known in the art, such as three color fluorescence immunocytometry. Monoclonal antibodies to a soluble human ZTNF9 polypeptide can also be used to mimic ligand/receptor coupling, resulting in activation or inactivation of the ligand/receptor pair. For instance, it has been demonstrated that cross-linking anti-soluble GP39 monoclonal antibodies inhibits signal from T cells to B cells (Noelle et al., Proc. Natl. Acad. Sci. USA 89:6650, 1992). Monoclonal antibodies to ZTNF9 can be used to determine the distribution, regulation and biological interaction of the ZTNF9 receptor/ZTNF9 ligand pair on specific cell lineages identified by tissue distribution studies, in particular, T cell lineages. Antibodies to ZTNF9 can also be used to detect secreted, soluble ZTNF9 in biological samples.

Antigenic epitope-bearing peptides and polypeptides contain at least four to ten amino acids, or at least ten to fifteen amino acids, or 15 to 30 amino acids of SEQ ID NO:2. Such epitope-bearing peptides and polypeptides can be produced by fragmenting an ZTNF9 polypeptide, or by chemical peptide synthesis, as described herein. Moreover, epitopes can be selected by phage display of random peptide libraries (see, for example, Lane and Stephen, Curr. Opin. Immunol. 5:268 (1993), and Cortese et al., Curr. Opin. Biotechnol. 7:616 (1996)). Standard methods for identifying epitopes and producing antibodies from small peptides that comprise an epitope are described, for example, by Mole, “Epitope Mapping,” in Methods in Molecular Biology, Vol. 10, Manson (ed.), pages 105-116 (The Humana Press, Inc. 1992), Price, “Production and Characterization of Synthetic Peptide-Derived Antibodies,” in Monoclonal Antibodies: Production, Engineering, and Clinical Application, Ritter and Ladyman (eds.), pages 60-84 (Cambridge University Press 1995), and Coligan et al. (eds.), Current Protocols in Immunology, pages 9.3.1 - 9.3.5 and pages 9.4.1 - 9.4.11 (John Wiley & Sons 1997).

ZTNF9 polypeptides can also be used to prepare antibodies that specifically bind to ZTNF9 epitopes, peptides or polypeptides. The ZTNF9 polypeptide or a fragment thereof serves as an antigen (immunogen) to inoculate an animal and elicit an immune response. One of skill in the art would recognize that antigenic, epitope-bearing polypeptides contain a sequence of at least 6, or at least 9, and at least 15 to about 30 contiguous amino acid residues of a ZTNF9 polypeptide (e.g., SEQ ID NO:2). Polypeptides comprising a larger portion of a ZTNF9 polypeptide, i.e., from 30 to 10 residues up to the entire length of the amino acid sequence are included. Antigens or immunogenic epitopes can also include attached tags, adjuvants and carriers, as described herein. Suitable antigens include the ZTNF9 polypeptides encoded by SEQ ID NO:2 from amino acid number I to amino acid number 87, or a contiguous 9 to 87 amino acid fragment thereof.

As an illustration, potential antigenic sites in ZTNF9 were identified using the Jameson-Wolf method, Jameson and Wolf, CABIOS 4:181, (1988), as implemented by the PROTEAN program (version 3.14) of LASERGENE (DNASTAR; Madison, Wis.). Default parameters were used in this analysis.

Suitable antigens include residue 20 to residue 40 of SEQ ID NO:2; residue 45 to residue 51 of SEQ ID NO:2; and residue 57 to residue 68 of SEQ ID NO:2. Hydrophilic peptides, such as those predicted by one of skill in the art from a hydrophobicity plot are also immunogenic. ZTNF9 hydrophilic peptides include peptides comprising amino acid sequences selected from the group consisting of: residue 20 to residue 39 of SEQ ID NO:2; and residue 42 to residue 69 of SEQ ID NO:2. Additionally, antigens can be generated to portions of the polypeptide which are likely to be on the surface of the folded protein. These antigens include: residue 21 to residue 26 of SEQ ID NO:2; and residue 30 to residue 35 of SEQ ID NO:2. Antibodies from an immune response generated by inoculation of an animal with these antigens can be isolated and purified as described herein. Methods for preparing and isolating polyclonal and monoclonal antibodies are well known in the art. See, for example, Current Protocols in Immunology, Cooligan, et al. (eds.), National Institutes of Health, John Wiley and Sons, Inc., 1995; Sambrook et al., Molecular Cloning: A Laboratory Manual, Second Edition, Cold Spring Harbor, N.Y., 1989; and Hurrell, J. G. R., Ed., Monoclonal Hybridoma Antibodies: Techniques and Applications, CRC Press, Inc., Boca Raton, Fla., 1982.

ZTNF9 ligand polypeptides and soluble ZTNF9 ligands may be used to identify and characterize receptors in the TNFR family. The receptor for ZTNF9 is likely an as yet unidentified TNFR, but it is possible that ZTNF9 may bind one of the known members of the TNFR family, such as TNF and lymphotoxin- a bind to the TNF receptor. Proteins and peptides of the present invention can be immobilized on a column and membrane preparations run over the column (Immobilized Affinity Ligand Techniques, Hermanson et al., eds., Academic Press, San Diego, Calif., 1992, 195-202). Proteins and peptides can also be radiolabeled (Methods in Enzymol., vol. 182, “Guide to Protein Purification”, M. Deutscher, ed., Acad. Press, San Diego, 1990, 721-37) or photoaffinity labeled (Brunner et al., Ann. Rev. Biochem. 62:483-514, 1993 and Fedan et al., Biochem. Pharmacol. 33:1167-80, 1984) and specific cell-surface proteins can be identified. The soluble ligand is useful in studying the distribution of receptors on tissues or specific cell lineages, and to provide insight into receptor/ligand biology. Application may also be made of the specificity of TNF ligands for their receptor as a mechanism by which to destroy receptor-bearing target cells. For example, toxic compounds may be coupled to ZTNF9 ligands, in particular to soluble ligands (Mesri et al., J. Biol. Chem. 268:4853-62, 1993). Examples of toxic compounds would include radiopharmaceuticals that inactivate target cells; chemotherapeutic agents such as doxorubicin, daunorubicin, methotrexate, and cytoxan; toxins, such as ricin, diphtheria, Pseudomonas exotoxin A and abrin; and antibodies to cytotoxic T-cell surface molecules.

The tissue specificity of ZTNF9 expression suggests a role in spermatogenesis, a process that is similar to the development of blood cells (hematopoiesis). Briefly, spermatogonia undergo a maturation process similar to the differentiation of hematopoietic stem cells. In view of the tissue specificity observed for ZTNF9, agonists and antagonists have enormous potential in both in vitro and in vivo applications. ZTNF9 polypeptides, agonists and antagonists may also prove useful in modulating spermatogenesis and thus aid in overcoming infertility. Antagonists are useful as research reagents for characterizing sites of ligand-receptor interaction. In vivo, ZTNF9 polypeptides, agonists or antagonists may find application in the treatment of male infertility or as a male contraceptive agents.

The ZTNF9 polypeptides, antagonists of agonists, of the present invention can also modulate sperm capacitation. Before reaching the oocyte or egg and initiating an egg-sperm interaction, the sperm must be activated. The sperm undergo a gradual capacitation, lasting up to 3 or 4 hours in vitro, during which the plasma membrane of the sperm head and the outer acrosomal membrane fuse to form vesicles that facilitate the release of acrosomal enzymes. The acrosomal membrane surrounds the acrosome or acrosomal cap which is located at the anterior end of the nucleus in the sperm head. In order for the sperm to fertilize egg the sperm must penetrate the oocyte. To enable this process the sperm must undergo acrosomal exocytosis, also known as the acrosomal reaction, and release the acrosomal enzymes in the vicinity of the oocyte. These enzymes enable the sperm to penetrate the various oocyte layers, (the cumulus oophorus, the corona radiata and the zona pellucida). The released acrosomal enzymes include hyaluronidase and proacrosin, in addition to other enzymes such as proteases. During the acrosomal reaction, proacrosin is converted to acrosin, the active form of the enzyme, which is required for and must occur before binding and penetration of the zona pellucida is possible. A combination of the acrosomal lytic enzymes and sperm tail movements allow the sperm to penetrate the oocyte layers. Numerous sperm must reach the egg and release acrosomal enzymes before the egg can finally be fertilized. Only one sperm will successfully bind to, penetrate and fertilize the egg, after which the zona hardens so that no other sperm can penetrate the egg (Zaneveld, in Male Infertility Chapter 11, Comhaire (Ed.), Chapman & Hall, London, 1996). Peptide hormones, such as insulin homologs are associated with sperm activation and egg-sperm interaction. For instance, capacitated sperm incubated with relaxin show an increased percentage of progressively motile sperm, increased zona penetration rates, and increased percentage of viable acrosome-reacted sperm (Carrell et al., Endocr. Res. 21:697-707, 1995). Localization of ZTNF9 to the testis suggests that the ZTNF9 polypeptides described herein play a role in these and other reproductive processes.

Accordingly, proteins of the present invention can have applications in enhancing fertilization during assisted reproduction in humans and in animals. Such assisted reproduction methods are known in the art and include artificial insemination, in vitro fertilization, embryo transfer and gamete intrafallopian transfer. Such methods are useful for assisting men and women who have physiological or metabolic disorders preventing natural conception or can be used to enhance in vitro fertilization. Such methods are also used in animal breeding programs, such as for livestock breeding and could be used as methods for the creation of transgenic animals. Proteins of the present invention can be combined with sperm, an egg or an egg-sperm mixture prior to fertilization of the egg. In some species, sperm capacitate spontaneously during in vitro fertilization procedures, but normally sperm capacitate over an extended period of time both in vivo and in vitro. It is advantageous to increase sperm activation during such procedures to enhance the likelihood of successful fertilization. The washed sperm or sperm removed from the seminal plasma used in such assisted reproduction methods has been shown to have altered reproductive functions, in particular, reduced motility and zona interaction. To enhance fertilization during assisted reproduction methods sperm is capacitated using exogenously added compounds. Suspension of the sperm in seminal plasma from normal subjects or in a “capacitation media” containing a cocktail of compounds known to activate sperm, such as caffeine, dibutyl cyclic adenosine monophosphate (dbcAMP) or theophylline, have resulted in improved reproductive function of the sperm, in particular, sperm motility and zonae penetration (Park et al., Am. J. Obstet. Gynecol. 158:974-9, 1988; Vandevoort et al., Mol. Repro. Develop. 37:299-304, 1993; Vandevoort and Overstreet, J. Androl. 16:327-33, 1995). The presence of immunoreactive relaxin in vivo and in association with cryopreserved semen, was shown to significantly increase sperm motility (Juang et al., Anim. Reprod. Sci. 20:21-9, 1989; Juang et al., Anim. Reprod. Sci. 22:47-53, 1990). Porcine relaxin stimulated sperm motility in cryopreserved human sperm (Colon et al., Fertil. Steril. 46:1133-39, 1986; Lessing et al., Fertil. Steril. 44:406-9, 1985) and preserved ability of washed human sperm to penetrate cervical mucus in vitro (Brenner et al., Fertil. Steril. 42:92-6, 1984). Polypeptides of the present invention can used in such methods to enhance viability of cryopreserved sperm, enhance sperm motility and enhance fertilization, particularly in association with methods of assisted reproduction.

In cases where pregnancy is not desired, ZTNF9 polypeptide or polypeptide fragments may function as germ-cell-specific antigens for use as components in “immunocontraceptive” or “anti-fertility” vaccines to induce formation of antibodies and/or cell mediated immunity to selectively inhibit a process, or processes, critical to successful reproduction in humans and animals. The use of sperm and testis antigens in the development of immunocontraceptives have been described (O'Hern et al., Biol Reprod. 52:311-39, 1995; Diekman and Herr, Am. J. Reprod. Immunol. 37:111-17, 1997; Zhu and Naz, Proc. Natl. Acad. Sci. USA 94:4704-9,1997). A vaccine based on human chorionic gonadotrophin (HCG) linked to a diphtheria or tetanus carrier was in clinical trials (Talwar et al., Proc. Natl. Acad. Sci. USA 91:8532-36, 1994). A single injection resulted in production of high titer antibodies that persisted for nearly a year in rabbits (Stevens, Am. J. Reprod. Immunol. 29:176-88, 1993). Such methods of immunocontraception using vaccines could include a ZTNF9 testes-specific protein or fragment thereof. The ZTNF9 protein or fragments can be conjugated to a carrier protein or peptide, such as tetanus or diphtheria toxoid. An adjuvant, as described above, can be included and the protein or fragment can be noncovalently associated with other molecules to enhance intrinsic immunoreactivity. Methods for administration and methods for determining the number of administrations are known in the art. Such a method might include a number of primary injections over several weeks followed by booster injections as needed to maintain a suitable antibody titer.

The polypeptides, antagonists, agonists, nucleic acid and/or antibodies of the present invention may be used in treatment of disorders associated with gonadal development, pubertal changes, fertility, neuralgia associated with reproductive phenomena, male sexual dysfunction, impotency, testicular cancer and dysfunction. The molecules of the present invention may used to modulate or to treat or prevent development of pathological conditions in such diverse tissue, including testis. In particular, certain syndromes or diseases may be amenable to such diagnosis, treatment or prevention. Moreover, natural functions, such as spermatogenesis, may be suppressed or controlled for use in birth control by molecules of the present invention.

Molecules expressed in the testis, such as ZTNF9 polypeptides, may modulate hormones, hormone receptors, growth factors, or cell-cell interactions, of the reproductive cascade or be involved in spermatogenesis, or testis development, would be useful as markers for cancer of reproductive organs and as therapeutic agents for hormone-dependent cancers, by inhibiting hormone-dependent growth and/or development of tumor cells. Human reproductive system cancers such as testicular and prostate cancers are common. Moreover, receptors for steroid hormones involved in the reproductive cascade are found in human tumors and tumor cell lines (breast, prostate, endometrial, ovarian, kidney, and pancreatic tumors) (Kakar et al., Mol. Cell. Endocrinol., 106:145-49, 1994; Kakar and Jennes, Cancer Letts., 98:57-62, 1995). Thus, expression of ZTNF9 in reproductive tissues suggests that polypeptides of the present invention would be useful in diagnostic methods for the detection and monitoring of reproductive cancers.

Diagnostic methods of the present invention involve the detection of ZTNF9 polypeptides in the serum or tissue biopsy of a patient undergoing analysis of reproductive function or evaluation for possible reproductive cancers, e.g., testicular or prostate cancer. Such polypeptides can be detected using immunoassay techniques and antibodies, described herein, that are capable of recognizing ZTNF9 polypeptide epitopes. More specifically, the present invention contemplates methods for detecting ZTNF9 polypeptides comprising:

exposing a test sample potentially containing ZTNF9 polypeptides to an antibody attached to a solid support, wherein said antibody binds to a first epitope of a ZTNF9 polypeptide;

washing the immobilized antibody-polypeptide to remove unbound contaminants;

exposing the immobilized antibody-polypeptide to a second antibody directed to a second epitope of a ZTNF9 polypeptide, wherein the second antibody is associated with a detectable label; and

detecting the detectable label. Altered levels of ZTNF9 polypeptides in a test sample, such as serum, semen, urine, sweat, saliva, biopsy, and the like, can be monitored as an indication of reproductive function or of reproductive cancer or disease, when compared against a normal control.

Additional methods using probes or primers derived, for example, from the nucleotide sequences disclosed herein can also be used to detect ZTNF9 expression in a patient sample, such as a blood, urine, semen, saliva, sweat, biopsy, tissue sample, or the like. For example, probes can be hybridized to tumor tissues and the hybridized complex detected by in situ hybridization. ZTNF9 sequences can also be detected by PCR amplification using cDNA generated by reverse translation of sample mRNA as a template (PCR Primer A Laboratory Manual, Dieffenbach and Dveksler, eds., Cold Spring Harbor Press, 1995). When compared with a normal control, both increases or decreases of ZTNF9 expression in a patient sample, relative to that of a control, can be monitored and used as an indicator or diagnostic for disease.

Moreover, the activity and effect of ZTNF9 on tumor progression and metastasis can be measured in vivo. Several syngeneic mouse models have been developed to study the influence of polypeptides, compounds or other treatments on tumor progression. In these models, tumor cells passaged in culture are implanted into mice of the same strain as the tumor donor. The cells will develop into tumors having similar characteristics in the recipient mice, and metastasis will also occur in some of the models. Tumor models include the Lewis lung carcinoma (ATCC No. CRL-1642) and B16 melanoma (ATCC No. CRL-6323), amongst others. These are both commonly used tumor lines, syngeneic to the C57BL6 mouse, that are readily cultured and manipulated in vitro. Tumors resulting from implantation of either of these cell lines are capable of metastasis to the lung in C57BL6 mice. The Lewis lung carcinoma model has recently been used in mice to identify an inhibitor of angiogenesis (O'Reilly MS, et al. Cell 79: 315-328,1994). C57BL6/J mice are treated with an experimental agent either through daily injection of recombinant protein, agonist or antagonist or a one time injection of recombinant adenovirus. Three days following this treatment, 10⁵ to 10⁶ cells are implanted under the dorsal skin. Alternatively, the cells themselves may be infected with recombinant adenovirus, such as one expressing ZTNF9, before implantation so that the protein is synthesized at the tumor site or intracellularly, rather than systemically. The mice normally develop visible tumors within 5 days. The tumors are allowed to grow for a period of up to 3 weeks, during which time they may reach a size of 1500-1800 mm³ in the control treated group. Tumor size and body weight are carefully monitored throughout the experiment. At the time of sacrifice, the tumor is removed and weighed along with the lungs and the liver. The lung weight has been shown to correlate well with metastatic tumor burden. As an additional measure, lung surface metastases are counted. The resected tumor, lungs and liver are prepared for histopathological examination, immunohistochemistry, and in situ hybridization, using methods known in the art and described herein. The influence of the expressed polypeptide in question, e.g., ZTNF9, on the ability of the tumor to recruit vasculature and undergo metastasis can thus be assessed. In addition, aside from using adenovirus, the implanted cells can be transiently transfected with ZTNF9. Moreover, purified ZTNF9 or ZTNF9-conditioned media can be directly injected in to this mouse model, and hence be used in this system. Use of stable ZTNF9 transfectants as well as use of induceable promoters to activate ZTNF9 expression in vivo are known in the art and can be used in this system to assess ZTNF9 induction of metastasis. For general reference see, O'Reilly MS, et al. Cell 79:315-328, 1994; and Rusciano D, et al. Murine Models of Liver Metastasis. Invasion Metastasis 14:349-361, 1995.

The invention also provides isolated and purified ZTNF9 polynucleotide probes. Such polynucleotide probes can be RNA or DNA. DNA can be either cDNA or genomic DNA. Polynucleotide probes are single or double-stranded DNA or RNA, generally synthetic oligonucleotides, but may be generated from cloned cDNA or genomic sequences and will generally comprise at least 16 nucleotides, more often from 17 nucleotides to 25 or more nucleotides, sometimes 40 to 60 nucleotides, and in some instances a substantial portion, domain or even the entire ZTNF9 gene or cDNA. The synthetic oligonucleotides of the present invention have at least 80% identity to a representative ZTNF9 DNA sequence (SEQ ID NO: 1) or its complements. Preferred regions from which to construct probes include the 5′ and/or 3′ coding sequences, receptor binding regions, extracellular, transmembrane and/or cytoplasmic domains, signal sequences and the like. Techniques for developing polynucleotide probes and hybridization techniques are known in the art, see for example, Ausubel et al., eds., Current Protocols in Molecular Biology, John Wiley and Sons, Inc., N.Y., 1991. For use as probes, the molecules can be labeled to provide a detectable signal, such as with an enzyme, biotin, a radionuclide, fluorophore, chemiluminescer, paramagnetic particle and the like, which are commercially available from many sources, such as Molecular Probes, Inc., (Eugene, Oreg.), and Amersham Corp., (Arlington Heights, Ill.), using techniques that are well known in the art.

Such probes can also be used in hybridizations to detect the presence or quantify the amount of ZTNF9 gene or mRNA transcript in a sample. ZTNF9 polynucleotide probes could be used to hybridize to DNA or RNA targets for diagnostic purposes, using such techniques such as fluorescent in situ hybridization (FISH) or immunohistochemistry.

Polynucleotide probes could be used to identify genes encoding ZTNF9-like proteins. For example, ZTNF9 polynucleotides can be used as primers and/or templates in PCR reactions to identify other novel members of the tumor necrosis factor family.

Such probes can also be used to screen libraries for related sequences encoding novel tumor necrosis factors. Such screening would be carried out under conditions of low stringency which would allow identification of sequences which are substantially homologous, but not requiring complete homology to the probe sequence. Such methods and conditions are well known in the art, see. for example, Sambrook et al., Molecular Cloning: A Laboratory Manual, Second Edition, Cold Spring Harbor, N.Y., 1989. Such low stringency conditions could include hybridization temperatures less than 42° C, formamide concentrations of less than 50% and moderate to low concentrations of salt. Libraries may be made of genomic DNA or cDNA.

Polynucleotide probes are also useful for Southern, Northern, or slot blots, colony and plaque hybridization and in situ hybridization. Mixtures of different ZTNF9 polynucleotide probes can be prepared which would increase sensitivity or the detection of low copy number targets, in screening systems.

ZTNF9 polypeptides may be used within diagnostic systems. Antibodies or other agents that specifically bind to ZTNF9 may be used to detect the presence of circulating receptor or ligand polypeptides. Such detection methods are well known in the art and include, for example, enzyme-linked immunosorbent assay (ELISA) and radioimmunoassay. Immunohistochemically labeled antibodies can be used to detect ZTNF9 ligand in tissue samples. ZTNF9 levels can also be monitored by such methods as RT-PCR, where ZTNF9 mRNA can be detected and quantified. Such methods could be used as diagnostic tools to monitor and quantify receptor or ligand polypeptide levels. The information derived from such detection methods would provide insight into the significance of ZTNF9 polypeptides in various diseases, and as a would serve as diagnostic methods for diseases for which altered levels of ZTNF9 are significant. Altered levels of ZTNF9 ligand polypeptides may be indicative of pathological conditions including cancer, autoimmune disorders, inflammation and immunodeficiencies.

The ZTNF9 polynucleotides and/or polypeptides disclosed herein can be useful as therapeutics, wherein ZTNF9 agonists and antagonists could modulate one or more biological processes in cells, tissues and/or biological fluids. Many members of the TNF family are expressed on lymphoid cells and mediate interactions between different immune cells. The homology of ZTNF9 with TNF suggests that ZTNF9 plays a role in regulation of the immune response, in particular the activation and regulation of lymphocytes. ZTNF9 polypeptides and ZTNF9 agonists would be useful as therapies for treating immunodeficiencies. The ZTNF9 polypeptides, ZTNF9 agonists and antagonists could be employed in therapeutic protocols for treatment of such autoimmune diseases as insulin dependent diabetes mellitus (IDDM), Crohn's Disease, muscular sclerosis (MS), myasthenia gravis (MG) and systemic lupus erythematosus.

ZTNF9 polypeptides and ZTNF9 agonists can be used to regulate anti-viral response, in treatments to combat infection and to provide relief from allergy symptoms. ZTNF9 polypeptides and ZTNF9 agonists can also be used to inhibit cancerous cell growth by acting as a mediator of cell apoptosis. ZTNF9 polypeptides and ZTNF9 agonists are also contemplated for use in regulation of certain carcinomas, such as lung carcinomas, small-cell cancers, squamous-cell carcinomas, large-cell carcinomas and adenocarcinomas.

ZTNF9 polynucleotides and polypeptides can be used as standards to calibrate in vitro cytokine assay systems or as standards within such assay systems. In addition, antibodies to ZTNF9 polypeptides could be used in assays for neutralization of bioactivity, in ELISA and ELISPOT assays, in Western blot analysis and for immunohistochemical applications. Various other cytokine proteins, antibodies and DNA are available from numerous commercial sources, such as R & D Systems, Minneapolis, Minn., for use in such methodologies.

The invention also provides antagonists, which either bind to ZTNF9 polypeptides or, alternatively, to a receptor to which ZTNF9 polypeptides bind, thereby inhibiting or eliminating the function of ZTNF9. Such ZTNF9 antagonists would include antibodies; oligonucleotides which bind either to the ZTNF9 polypeptide or to its receptor; natural or synthetic analogs of ZTNF9 polypeptides which retain the ability to bind the receptor but do not result in either ligand or receptor signaling. Such analogs could be peptides or peptide-like compounds. Natural or synthetic small molecules, which bind to receptors of ZTNF9 polypeptides and prevent signaling, are also contemplated as antagonists. As such, ZTNF9 antagonists would be useful as therapeutics for treating certain disorders where blocking signal from either a ZTNF9 ligand or receptor would be beneficial.

Antagonists would have additional therapeutic value for treating chronic inflammatory diseases, in particular to lessen joint pain, swelling, anemia and other associated symptoms. Antagonists are also useful in preventing bone resorption. They could also find use in treatments for rheumatoid arthritis and systemic lupus erythematosius. Antagonists would also find use in treating septic shock.

ZTNF9 polypeptides and ZTNF9 polypeptide antagonists can be employed in the study of effector functions of T lymphocytes, in particular T lymphocyte activation and differentiation. Also in T helper functions in mediating humoral or cellular immunity. ZTNF9 polypeptides and ZTNF9 polypeptide antagonists are also contemplated as useful research reagents for characterizing ligand-receptor interactions.

The invention also provides nucleic acid-based therapeutic treatment. If a mammal has a mutated or lacks a ZTNF9 gene, the ZTNF9 gene can be introduced into the cells of the mammal. In one embodiment, a gene encoding a Ztnf9 polypeptide is introduced in vivo in a viral vector. Such vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, but are not limited to, a defective herpes virus 1 (HSV1) vector (Kaplitt et al., Molec. Cell. Neurosci. 2:320-30, 1991), an attenuated adenovirus vector, such as the vector described by Stratford-Perricaudet et al. (J. Clin. Invest. 90:626-30, 1992), and a defective adeno-associated virus vector (Samulski et al., J. Virol. 61:3096-101, 1987; Samulski et al., J. Virol. 63:3822-8, 1989).

In another embodiment, the gene can be introduced in a retroviral vector, e.g., as described in Anderson et al., U.S. Pat. No. 5,399,346; Mann et al., Cell 33:153, 1983; Temin et al., U.S. Pat. No. 4,650,764; Temin et al., U.S. Pat. No. 4,980,289; Markowitz et al., J. Virol. 62:1120, 1988; Temin et al., U.S. Pat. No. 5,124,263; Dougherty et al., WIPO Publication WO 95/07358; and Kuo et al., Blood 82:845-52, 1993.

Alternatively, the vector can be introduced by lipofection in vivo using liposomes. Synthetic cationic lipids can be used to prepare liposomes for in vivo transfection of a gene encoding a marker (Felgner et al., Proc. Natl. Acad. Sci. USA 84:7413-17, 1987; and Mackey et al., Proc. Natl. Acad. Sci. USA 85:8027-31, 1988). The use of lipofection to introduce exogenous genes into specific organs in vivo has certain practical advantages. Molecular targeting of liposomes to specific cells represents one area of benefit. It is clear that directing transfection to particular cells represents one area of benefit. It is clear that directing transfection to particular cell types would be particularly advantageous in a tissue with cellular heterogeneity, such as the pancreas, liver, kidney, and brain. Lipids may be chernically coupled to other molecules for the purpose of targeting. Targeted peptides, e.g., hormones or neurotransmitters, and proteins such as antibodies, or non-peptide molecules could be coupled to liposomes chemically.

It is possible to remove the cells from the body and introduce the vector as a naked DNA plasmid and then re-implant the transformed cells into the body. Naked DNA vector for gene therapy can be introduced into the desired host cells by methods known in the art, e.g., transfection, electroporation, microinjection, transduction, cell fusion, DEAE dextran, calcium phosphate precipitation, use of a gene gun or use of a DNA vector transporter (see, for example, Wu et al., J. Biol. Chem. 267:963-7, 1992; Wu et al., J. Biol. Chem. 263:14621-24, 1988).

The ZTNF9 polypeptides are also contemplated for pharmaceutical use. Pharmaceutically effective amounts of ZTNF9 polypeptides, agonists or ZTNF9 antagonists of the present invention can be formulated with pharmaceutically acceptable carriers for parenteral, oral, nasal, rectal, topical, transdermal administration or the like, according to conventional methods. Formulations may further include one or more diluents, fillers, emulsifiers, preservatives, buffers, excipients, and the like, and may be provided in such forms as liquids, powders, emulsions, suppositories, liposomes, transdermal patches and tablets, for example. Slow or extended-release delivery systems, including any of a number of biopolymers (biological-based systems), systems employing liposomes, and polymeric delivery systems, can also be utilized with the compositions described herein to provide a continuous or long-term source of the ZTNF9 polypeptide or antagonist. Such slow release systems are applicable to formulations, for example, for oral, topical and parenteral use. The term “pharmaceutically acceptable carrier” refers to a carrier medium which does not interfere with the effectiveness of the biological activity of the active ingredients and which is not toxic to the host or patient. One skilled in the art may formulate the compounds of the present invention in an appropriate manner, and in accordance with accepted practices, such as those disclosed in Remington's Pharmaceutical Sciences, Gennaro (ed.), Mack Publishing Co., Easton, Pa. 1990.

As used herein a “pharmaceutically effective amount” of a ZTNF9 polypeptide, agonist or antagonist is an amount sufficient to induce a desired biological result. The result can be alleviation of the signs, symptoms, or causes of a disease, or any other desired alteration of a biological system. For example, an effective amount of a ZTNF9 polypeptide or antagonist is that which provides either subjective relief of symptoms or an objectively identifiable improvement as noted by the clinician or other qualified observer. It may also be an amount which results in reduction of serum Ca++levels or an inhibition of osteoclast size and number in response to treatment for bone resorption. Other such examples include reduction in acetylcholine antibody levels, a decrease in muscle weakness during treatment for myasthenia gravis; or other beneficial effects. Effective amounts of ZTNF9 for use in treating muscular sclerosis (MS) would result in decrease in muscle weakness, and/or a reduction in frequency of MS exacerbation. In EAE mouse model measurements, EAE grades, of clinical signs of disease, such as limp tail or degree of paralysis are made. For rheumatoid arthritis, such indicators include a reduction in inflammation and relief of pain or stiffness, in animal models indications would be derived from macroscopic inspection of joints and change in swelling of hind paws. Effective amounts of the ZTNF9 polypeptides can vary widely depending on the disease or symptom to be treated. The polypeptides, polynucleotides, and antibodies of the present invention, as well as fragments thereof will be useful in treating diseases including, osteoporosis, Paget's disease, hyperparathyroidism, arthrtitis, osteopetrosis, osteopenia, diseases related to skeletal integrity and calcium metabolism, and infertility. Similarly, the molecules of the present invention can be used as a method of contraceptive, or of enhancing fertility and spermatogenesis.

The amount of the polypeptide to be administered and its concentration in the formulations, depends upon the vehicle selected, route of administration, the potency of the particular polypeptide, the clinical condition of the patient, the side effects and the stability of the compound in the formulation. Thus, the clinician will employ the appropriate preparation containing the appropriate concentration in the formulation, as well as the amount of formulation administered, depending upon clinical experience with the patient in question or with similar patients. Such amounts will depend, in part, on the particular condition to be treated, age, weight, and general health of the patient, and other factors evident to those skilled in the art. Typically a dose will be in the range of 0.1-100 mg/kg of subject. Doses for specific compounds may be determined from in vitro or ex vivo studies in combination with studies on experimental animals. Concentrations of compounds found to be effective in vitro or ex vivo provide guidance for animal studies, wherein doses are calculated to provide similar concentrations at the site of action. Doses determined to be effective in experimental animals are generally predictive of doses in humans within one order of magnitude.

The dosages of the present compounds used to practice the invention include dosages effective to result in the desired effects. Estimation of appropriate dosages effective for the individual patient is well within the skill of the ordinary prescribing physician or other appropriate health care practitioner. As a guide, the clinician can use conventionally available advice from a source such as the Physician's Desk Reference, 48^{th} Edition, Medical Economics Data Production Co., Montvale, N.J. 07645-1742 (1994).

Preferably the compositions are presented for administration in unit dosage forms. The term “unit dosage form” refers to physically discrete units suitable as unitary dosed for human subjects and animals, each unit containing a predetermined quantity of active material calculated to produce a desired pharmaceutical effect in association with the required pharmaceutical diluent, carrier or vehicle. Examples of unit dosage forms include vials, ampules, tablets, caplets, pills, powders, granules, eyedrops, oral or ocular solutions or suspensions, ocular ointments, and oil-in-water emulsions. Means of preparation, formulation and administration are known to those of skill, see generally Remington's Pharmaceutical Science 15^{th} ed., Mack Publishing Co., Easton, Pa. (1990).

The invention is further illustrated by the following non-limiting examples.

### EXAMPLES

Example 1

Identification of the DNA Sequence

The novel ZTNF9 polypeptide-encoding polynucleotides of the present invention were initially identified by querying a database of partial sequences. The cDNA sequences identified from the query were not full-length. Thus, the full-length cDNA sequence was identified by PCR using oligonucleotide primers ZC40264 (SEQ ID NO:) and ZC40267 (SEQ ID NO:), designed to the 5′ and 3′ , untranslated regions of the gene, respectively. The gene was amplified from a testis CDNA library using the following thermalcycler conditions: one cycle at 94° C. for 4 minutes; followed by thirty cycles at 94° C. for 30 seconds, 58° C. for 30 seconds, 72° C. for 2 minutes, followed by one cycle at 72° C. for 7 minutes, followed by a 4° C. hold. The DNA fragment was purified using Gel Extraction Kit (Qiagen, Chatsworth, Calif.) and subcloned into pCR-4-TOPO using TOPO TA Cloning Kit for Sequencing(Invitrogen). The polynucleotide sequence of the insert corresponding to the cDNA clone was sequenced resulting in the polynucleotide sequence shown in SEQ ID NO:1. The deduced amino acid sequence of the insert was determined to be full-length and is shown in SEQ ID NO:2. This polypeptide, and the polynucleotides encoding it, were identified as a novel tumor necrosis factor.

Example 2

Tissue Distribution of Human Ztnf9 in Tissue Panels using PCR

A panel of cDNA samples from human tissues was screened for znssp8 expression using PCR. The panel was made in-house and contained 94 cDNA samples from marathon cDNA and cDNA samples from various normal and cancerous human tissues and cell lines, including adrenal gland, bone marrow, bladder, fetal brain, islet, brain, prostate, cervix, colon, testis, thyroid, fetal heart, fetal kidney, fetal liver, fetal lung, fetal muscle, fetal skin, prostate smooth muscle, heart, kidney, heart, liver, pituitary, lung, placenta, lymph node, salivary gland, melanoma, pancreas, pituitary, placenta, prostate, rectum, salivary gland, skeletal muscle, small intestine, spinal cord, spleen, stomach, testis, thymus, thyroid, trachea, uterus, esophagus tumor, gastric tumor, kidney tumor, liver tumor, lung tumor, ovarian tumor, rectal tumor, uterus tumor, RPMI #1788 (ATCC. # CCL-156), W138 (ATCC. # CCL-75, ARIP (ATCC. # CRL-1674-rat), HaCat-human keratinocytes, HPV (ATCC. # CRL-2221), CD3+selected PBMC's (stimulated), K562 (ATCC. # CCL-243), HPVS (ATCC. # CRL-2221)-selected, HL60 (ATCC. # CCL-240), platelet, renal mesangial, T-cell, neutrophil, MPC, Hut-102 (ATCC. # TIB-162), endothelial, HepG2 (ATCC. # HB-8065), fibroblast, and E. Histo. The cDNA samples came from in-house libraries or marathon cDNA preparations of RNA that were prepared in-house, or from a commercial supplier such as Clontech (Palo Alto, Calif.) or Invitrogen (Carlsbad, Calif.). The marathon cDNAs were made using the Marathon-Ready™ Kit (Clontech, Palo Alto, Calif.) and standardized to ensure an equal amount of cDNA was placed into each well. To assure quality of the panel samples, three tests for quality control (QC) were run: (1) To assess the RNA quality used for the libraries, the in-house cDNAs were tested for average insert size by PCR with vector oligos that were specific for the vector sequences for an individual cDNA library; (2) Standardization of the concentration of the cDNA in panel samples was achieved using standard PCR methods to amplify full length alpha tubulin or G3PDH cDNA; and (3) a sample was sent to sequencing to check for possible ribosomal or mitochondrial DNA contamination. The panel was set up in a 96-well format that included a human genomic DNA (Clontech, Palo Alto, Calif.) positive control sample. Each well contained approximately 0.2-100 pg/μl of cDNA. The PCR reactions were set up using oligos ZC23674 (SEQ ID NO: ) and ZC24776 (SEQ ID NO:), TaKaRa Ex Taq™ (TAKARA Shuzo Co LTD, Biomedicals Group, Japan), and Rediload dye (Research Genetics, Inc., Huntsville, Ala.). The amplification was carried out as follows: 1 cycle at 94° C. for 3 minutes, 35 cycles of 94° C. for 30 seconds, 58° C. for 30 seconds and 72° C. for 30 seconds, followed by 1 cycle at 72° C. for 5 minutes. About 10 μl of the PCR reaction product was subjected to standard Agarose gel electrophoresis using a 4% agarose gel. The correct predicted DNA fragment size was observed in testis cDNA library.

The DNA fragments for testis cDNA were excised and purified using a Gel Extraction Kit (Qiagen, Chatsworth, Calif.) according to manufacturer's instructions. Fragments were confirmed by sequencing to show that they were indeed ztnf9.

Example 3

Baculovirus Expression

An expression vector, pzBV37L:sNFzTNF9 was designed to express FlagzTNF9 polypeptide in insect cells. PzBV37L:sNFzTNF9 includes an upstream n-terminal flag epitope tag. This construct can be used to express a flag-tagged zTNF9 polypeptide.

A. Construction of pzBV37L:sNFzTNF9

A 254 bp fragment of FlagzTNF9 containing Bspel and Xbal restriction sites on the 5′ and 3′ ends, respectively, was generated by two rounds of PCR amplification from a zTNF9 cDNA containing template vector #101007. Primers #zc40918 and #zc40920 were used in the first round and primers #zc40919 and #zc40920 in the second round. For the first round of PCR, reaction conditions were as follows: utilized the Expand High Fidelity PCR System (Boehringer Mannheim cat # 1 732 641) for a 100 μl volume reaction: 1 cycle at 94° C. for 4 minutes; 30 cycles of 94° C. for 30 seconds, 50° C. for 30 seconds, and 72° C. for 45 seconds; I cycle at 72° C. for 4 min; followed by 4° C. soak. Five μl of the first round reaction mix was visualized by gel electrophoresis (1% NuSieve agarose). Once the presence of a correct size PCR product was confirmed, the second round of PCR was set up using 2 μl of the first round reaction as template. Conditions of the second reaction were the same as the first. Five μl of the second round PCR was visualized by gel electrophoresis (1% NuSieve agarose). The remainder of the reaction mix was purified via Qiagen PCR purification kit as per manufacturer's instructions and eluted in 30μl water. The cDNA was digested in a 36 μl vol. using Bspe1 and Xba1 (New England Biolabs, Beverly, Mass.) in appropriate buffer conditions at 37° C. The digested PCR product band was run through a 1% agarose TAE gel, excised and extracted using a QLAquick™ Gel Extraction Kit (Qiagen, Cat. No. 28704) and eluted in 30μl of water. The digested FlagzTNF9 PCR was ligated into the Multiple Cloning Site of vector pZBV37L at the Bspe1 and Xba1 sites. The pZBV37L vector is a modification of the pFastBac1™ (Life Technologies) expression vector, wherein the polyhedron promoter has been removed and replaced with the late-activating Basic Protein Promoter and the EGT (ecdysteroid UDP glycosyltransferase) leader signal sequence upstream of the MCS. Five μl of the Bspe1-Xba1 digested FlagzTNF9 PCR fragment and approximately 50 ng of the corresponding pZBV37L vector were ligated overnight at 16° C. in a 20μl volume in appropriate buffer conditions. Five μl of the ligation mix was transformed into 50 μl of ElectoMAX™ DH12STM cells (Life Technologies, Cat. No. 18312-017) by electroporation at 400 Ohms, 2 kV and 25 μF in a 2 mm gap electroporation cuvette (BTX, Model No. 620). The transformed cells were diluted in 500 μl of LB media, outgrown for 1 hr at 37° C., and 20 μl of the dilution were plated onto Luria Agar plates containing 100 μg/ml ampicillin. Clones were analyzed by PCR and positive clones were selected, plated and submitted for sequencing. Once proper sequence was confirmed, 25 ng of positive clone DNA was transformed into 70 μl DH10Bac™ Max Efficiency® competent cells (GIBCO-BRL Cat. No. 10361-012) by heat shock for 45 seconds in a 42° C. heat block. The transformed DH10Bac™ cells were diluted in 600 μl SOC. media (2% Bacto Tryptone, 0.5% Bacto Yeast Extract, 10 ml 1M NaCl, 1.5 mM KCl, 10 mM MgCl₂, 10 mM MgSO₄ and 20 mM glucose), outgrown for 1 hr at 37° C., and 50 μl were plated onto Luria Agar plates containing 50 μg/ml kanamycin, 7 μg/ml gentamicin, 10 μg/ml tetracycline, 40 μg/mL IPTG and 200 μg/mL Bluo Gal. The plates were incubated for 48 hours at 37° C. A color selection was used to identify those cells having transposed viral DNA (referred to as a “bacmid”). Those colonies, which were white in color, were picked for analysis. White, positive colonies (containing the desired bacmid) were selected for outgrow. The bacmid DNA was then isolated and purified. This bacmid DNA was used to transfect Spodoptera Frugiperda (Sf9) cells.

B. Transfection

15 Sf9 cells were seeded at 1×10⁶ cells per well in a 6-well plate and allowed to attach for 1 hour at 27° C. Approximately 5 μg of bacmid DNA were diluted with 100 μl Sf-900 II SFM (Life Technologies). Fifteen μl of Lipofectamine™ Reagent (Life Technologies, Cat. No. 18324-012) were diluted with 85 μl Sf-900 II SFM. The bacmid DNA and lipid solutions were gently mixed and incubated at room temperature for 45 minutes. Eight hundred μl of Sf-900 II SFM was added to the lipid-DNA mixture. The media was aspirated from the well and the lml of DNA-lipid mix added to the cells. The cells were incubated at 27° C. (90% humidity) overnight. The DNA-lipid mix was aspirated and 2 ml of fresh Sf-900 II media was added to each plate. The plates were incubated at 27° C., 90% humidity, for approximately 7 days after which the virus was harvested.

C. Amplification

Sf9 cells were seeded at 1×10⁶ cells per well in a 6-well plate in 2 mls SF-900II. 500 μl of virus from the transfection plate were placed in the well and the plate was incubated at 27° C., 90% humidity, for 96 hours after which the virus was harvested (primary amplification).

A second round of amplification proceeded as follows: Sf9 cells were seeded at 1×10⁶ cells per well in a 6-well plate in 2 ml SF-900II. One hundred μl of virus from the primary amplification plate were placed in the well and the plate was incubated at 27° C., 90% humidity, for 144 hours after which the virus was harvested (Secondary amplification).

An additional round of amplification was performed (3^{rd} round amp.) Sf9 cells were grown in 50 ml Sf-900 II SFM in a 250 ml shake flask to an approximate density of 1×10⁶ cells/ml. They were then infected with 1 mL of the viral stock from the above plate and incubated at 27° C. for 4 days after which time the virus was harvested.

This viral stock was titered by a growth inhibition curve and the titer culture that indicated an MOI of 1 was allowed to proceed for a total of 48 hours. The supernatant was analyzed via Western blot using a murine primary monoclonal antibody specific for the n-terminal Flag epitope, along with an HRP-conjugated Goat anti-Murine secondary antibody. Results indicated a band of approximately 9 kDa. Supernatant was also provided for activity analysis.

A large viral stock was then generated by the following method: Sf9 cells were grown in IL Sf-900 II SFM in a 2800 ml shake flask to an approximate density of 1×10⁶ cells/ml. They were then infected with 10 ml of the viral stock from the 3^{rd} round amplification and incubated at 27° C. for 96 hrs, after which time the virus was harvested.

Larger scale infections were completed to provide material for downstream purification.

Example 4

Analysis of Mouse Tibias

Ten-week-old C57BL/6J mice are treated with vehicle or ztnf9 protein (50 μg twice per day i.p., total 100 μg/day/animal) for 14 days. The mice are initially matched for age and body weight. After 14 days, the animals are sacrificed and the tibias are removed. Tibial bone samples are fixed in 10% neutral buffered formalin, decalcified in 5% formic acid with 10% sodium citrate, washed in tap water, dehydrated in a series of 70%-100% ethanol, and embedded in glycol methacrylate. The proximal end of the tibia (about 5 mm long) is cut frontally at 5 μm, stained for tartrate-resistant acid phosphatase (TRAP) activity, and counter-stained with methyl green and thionin for identification of bone cells.

Osteoblasts are identified by central negative (clear) Golgi area, eccentric nucleus, and the strong basophilic counter-stain of methyl green and thionin, while osteoclasts by TRAP stain, multinucleation, and non-uniform shape. The following bone parameters are evaluated for histomorphometric changes.

1) Growth plate activity: width measured every 50-100 μm at 42× magnification to determine the growth plate activity.

2) Number of endocortical osteoblasts: measured along one side of endocortical surface at 212× magnification.

3) Endocortical osteoblast size: measured using all the osteoblasts counted in the vehicle-treated mice or 50 osteoblasts randomly selected in the ztnf-treated mice at 424× magnification.

4) Number of endosteal osteoblasts: measured along the endosteal surface of cancellous bone in the metaphysis at 212× magnification in a zone area 0.62-3.10 mm distal to the growth plate.

5) Number of endosteal osteoclasts: measured simultaneously when endosteal osteoblast counts were taken.

6) Percentage of cancellous bone (bone volume/tissue volume, BV/TV): calculated from cancellous bone area per referent tissue area, and measured in the same reference areas where endosteal osteoblast and osteoclast counts are taken.

A significant change the number of endosteal osteoclasts, would suggest that bone resorption levels are altered.

Example 5

Calvarial Assay

The effects of ztnf9 polypeptides on bone growth are tested in ten-week-old CD-1 male mice by injecting ztnf9 protein into the subcutaneous tissue over the calvarium of the mice. Doses ranging from 0.001-5.0 mg/mouse are given three times daily for five days.

After 14 days, the mice are sacrificed and calvarial bone growth is measured by histomorphometry. Parameters measured are as described in Example 4.

Example 6

Ovariectomized Rat Assay

The ovariectomized rat is accepted as an animal model of human post-menopausal osteoporosis. To assess the effects of systemic administration of ztnf9 protein on skeletal tissues in an animal model of acute bone loss related to estrogen deficiency similar to that seen in post-menopausal women, female normal rats are either sham-operated or surgically ovariectomized. Seven days after surgery, treatment is begun, administering either vehicle, ztnf9 protein or estrogen (160 μg/kg, subcutaneously). Prior to sacrificing the animals, single doses of tetracycline or demeclocycline are administered to assess bone formation and mineralization. Tibias and lumbar vertebrae are removed, fixed, processed and analyzed as described in Example 1.

Example 7

Histomorphometric Examination of ztnf9 Treated Mice

Thirteen-week-old mice are weight-matched and treated with vehicle or ztnf9 for 14 or 28 days.

Both vehicle (0.007 mM borate in PBS) and ztnf9 are given twice daily by i.p. injections (ztnf9 50 μg 2×/day, total 100 μg). Mice are given food and water ad libitum. Calcein injections (15 mg/kg body weight) are given 9 and 2 days before sacrifice to label the newly formed bone for assessment of dynamic bone changes. All animals are sacrificed at the end of day 28. Tibial bone samples are fixed in 70% ethanol and embedded in methyl methacrylate without decalcification. The proximal end of the tibia (about 5 mm long) is cut parasagittally at 5 and 10 μm. Ten-μm sections are mounted without staining for evaluation of calcein labels, while 5-μm sections are stained for tartrate-resistant acid phosphatase (TRAP) activity and counter-stained with methyl green and thionin for identification of bone cells. Osteoblasts are identified by central negative (clear) Golgi area, eccentric nucleus, and the strong basophilic stain, while osteoclasts by TRAP stain, multinucleation, and non-uniform shape.

The following bone sites are evaluated for histomorphometric changes:

A. Endocortical Bone

Endocortical bone is evaluated at both the anterior and the posterior region within 3.7 mm below the growth plate at 90× magnification. In general, bone-forming activity can differ significantly at various sites within the bone, with greater activity at the posterior than the anterior region of the bone site evaluated.

B. Metaphysis

Because changes in bone cell activities following ztnf9 treatment could differ in the cancellous bone directly below (primary spongiosa) and further away from the growth plate (secondary spongiosa), histomorphometric bone changes are evaluated at both sites.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

We claim:

1. An isolated polypeptide comprising a sequence of amino acid residues from amino acid residue 62 to amino acid residue 81 as shown in SEQ ID NO:2.

2. The isolated polypeptide according to claim 1, wherein the polypeptide further comprises an amino acid sequence selected from the group consisting of:
a) a polypeptide comprising a sequence of amino acids from amino acid residue 20 to amino acid residue 87 as shown in SEQ ID NO:2;
b) a polypeptide that is at least 80% identical to a);
c) a polypeptide that is at least 85% identical to a);
d) a polypeptide that is at least 90% identical to a);
e) a polypeptide that is at least 95% identical to a); and
f) a polypeptide that is at least 99% identical to a).

3. The isolated polypeptide according to claim 2, wherein the polypeptide comprises the amino acid sequence from amino acid 20 to amino acid 87 as shown in SEQ ID NO:2.

4. The isolated polypeptide according to claim 2 wherein residues 62 through 81 as shown in SEQ ID NO:2 bind a tumor necrosis factor receptor.

5. The isolated polypeptide according to claim 4, wherein the polypeptide further comprises a linker region adjacent to the N-terminal of the residues 62 through 81.

6. The isolated polypeptide according to claim 5, wherein the polypeptide further comprises a transmembrane domain separated from residues 62 through 81 by the linker region.

7. The isolated polypeptide according to claim 6, wherein the polypeptide further comprises a cytoplasmic region separated from residues 62 through 81 by the transmembrane domain and linker region.

8. The isolated polypeptide according to claim 1, wherein the polypeptide further comprises an amino acid sequence selected from the group consisting of:
a) a polypeptide comprising a sequence of amino acids from amino acid residue 20 to amino acid residue 87 as shown in SEQ ID NO:2;
b) a polypeptide that is at least 80% identical to a);
c) a polypeptide that is at least 85% identical to a);
d) a polypeptide that is at least 90% identical to a);
e) a polypeptide that is at least 95% identical to a); and
f) a polypeptide that is at least 99% identical to a).

9. An isolated polypeptide according to claim 2, covalently linked to a moiety selected from the group consisting of affinity tags, toxins, radionucleotides, enzymes and fluorophores.

10. An isolated polypeptide according to claim 9, further comprising a proteolytic cleavage site between said polypeptide and said moiety.

11. A fusion protein consisting essentially of a first portion and a second portion joined by a peptide bond, said first portion consisting essentially of a polypeptide selected from the group consisting of:
a) the polypeptide according to claim 3;
b) a polypeptide having a sequence of amino acid residues that are at least 80% identical to (a);
wherein the second portion consisting essentially of a second polypeptide.

12. An antibody that specifically binds to an epitope of a polypeptide of SEQ ID NO:2.

13. The antibody according to claim 12, wherein the antibody is a monoclonal antibody.

14. A method of producing an antibody comprising the following steps in order:
inoculating an animal with a polypeptide selected from the group consisting of:
a) a polypeptide consisting of amino acid residues 68 to 82 as shown in SEQ ID NO:2;
b) a polypeptide consisting of amino acid residues 20 to 87 as shown in SEQ ID NO:2; and
c) a polypeptide consisting of amino acid residues to 87 as shown in SEQ ID NO:2;
wherein the polypeptide elicits an immune response in the animal to produce the antibody;
and isolating the antibody from the animal;
wherein the antibody specifically binds to the amino acid sequence of SEQ ID NO:2 from amino acid number 1 to amino acid number 87.

15. A binding protein that specifically binds to an epitope of the polypeptide according to claim 3.

16. An isolated polynucleotide encoding the amino acid sequence from amino acid residue 62 to amino acid residue 81 as shown in SEQ ID NO:2.

17. The isolated polynucleotide according to claim 16, wherein the polypeptide further comprises a polypeptide from amino residue 20 to amino acid residue 87 as shown in SEQ ID NO:2.

18. The isolated polynucleotide according to claim 17, wherein the polypeptide further comprises a polypeptide from amino residue 1 to amino acid residue 87 as shown in SEQ ID NO:2.

19. The isolated polynucleotide according to claim 16, wherein the polypeptide further comprises a linker region adjacent to the N-terminal of amino acid residues 62 through 81.

20. The isolated polynucleotide according to claim 19, wherein the polypeptide further comprises a transmembrane domain separated from amino acid residues 62 through 81 by the linker region.

21. The isolated polypeptide according to claim 20, wherein the polypeptide further comprises a cytoplasmic region separated from amino aicd residues 62 through 81 by a transmembrane domain and a linker region.

22. An expression vector comprising the following operably linked elements:
a transcription promoter;
a DNA segment encoding the polypeptide according to claim 2, wherein said polypeptide is a tumor necrosis factor; and
a transcription terminator.

23. The expression vector according to claim 22, wherein said DNA segment encodes a polypeptide further comprising an affinity tag.

24. A cultured cell into which has been introduced the expression vector according to claim 23.

25. A method of producing a polypeptide comprising:
culturing the cell according to claim 24, whereby said cell expresses said polypeptide encoded by said DNA segment; and
recovering said expressed polypeptide.

26. A method for detecting a genetic abnormality in a patient, comprising:
obtaining a genetic sample from a patient;
incubating the genetic sample with a polynucleotide comprising at least 14 contiguous nucleotides of SEQ ID NO: 1 or the complement of SEQ ID NO: 1, under conditions wherein said polynucleotide will hybridize to complementary polynucleotide sequence, to produce a first reaction product;
comparing said first reaction product to a control reaction product, wherein a difference between said first reaction product and said control reaction product is indicative of a genetic abnormality in the patient.

27. A method for the treatment of a mammal having a need for a ZTNF9 polypeptide comprising administering to said mammal a pharmaceutically effective amount of the polypeptide selected from the group consisting of:
a) a polypeptide comprising a sequence of amino acids from amino acid residue 20 to amino acid residue 87 of SEQ ID NO:2; and
b) a polypeptide having a sequence of amino acid residues that are at least 80% identical to (a).

28. The method according to claim 27, wherein the treatment is for abnormalities in bone remodeling, a fertility disorders, or arthritis.

29. A method for the treatment of a mammal having a need for an antagonist of a ZTNF9 polypeptide comprising administering to said mammal a pharmaceutically effective amount of an antagonist of a polypeptide selected from the group consisting of:
a) a polypeptide comprising a sequence of amino acids from amino acid residue 20 to amino acid residue 87 of SEQ ID NO:2;
b) a polypeptide having a sequence of amino acid residues that are at least 80% identical to (a).

30. The method according to claim 2, wherein the mammal is treated for abnormalities in bone remodeling, a fertility disorders, or arthritis.
